(19) United States
(12) Patent Application Publication
(10) Pub. No.: US 2009/0214101 A1
(43) Pub. Date: Aug. 27, 2009

(54) Targeted Nanostructures for Cellular Imaging

(76) Inventors: Lon J. Wilson, Houston, TX (US); Jared M. Ashcroft, Oakland, CA (US); Michael G. Rosenblum, Sugar Land, TX (US)

Correspondence Address: BAKER BOTTS, LLP, 910 LOUISIANA, HOUSTON, TX, 77002-4995, US

(21) Appl. No.: 12/325,849

(22) Filed: Dec. 1, 2008

Related U.S. Application Data
(63) Continuation of application No. PCT/US2007/070234, filed on Jun. 1, 2007.
(60) Provisional application No. 60/803,641, filed on Jun. 1, 2006.

(51) Int. Cl. G06K 9/00; C07K 16/18; G01N 33/483

(52) U.S. Cl. 382/133; 530/391.1; 252/408.1; 977/735; 977/734; 977/773

(57) ABSTRACT

Compositions and methods related to targeted carbon nanostructures. More particularly, targeted carbon nanostructures comprising: a Cₙ, a cross-linker, and a targeting agent, wherein Cₙ refers to a fullerene moiety or nanotube comprising n carbon atoms. One example of a method may involve a method for imaging comprising: contacting a targeted carbon nanostructure and a cell; allowing the cell to internalize the carbon nanostructure; and detecting the presence of internalized carbon nanostructures.

### CROSS-REFERENCE TO RELATED APPLICATIONS

This application is a continuation of International Application No. PCT/US2007/70234, filed Jun. 1, 2007, which claims the benefit of U.S. Provisional Application Ser. No. 60/803,641, filed Jun. 1, 2006, both of which are incorporated in this application by reference.

### BACKGROUND

Fullerene (C₆₀) materials have been studied extensively for use in nanomedicine and show great promise. Water-soluble C₆₀ derivatives are now commonplace and the discovery that water-soluble C₆₀ derivatives can cross cell membranes and even produce transfection has accelerated interest in utilizing C₆₀ for diagnostic and therapeutic medicine. Further, several water-soluble C₆₀ derivatives have demonstrated acceptable cytotoxicity for drug-delivery applications.

Similarly, interest in medical applications for carbon nanotubes is growing. Thus far, solubility properties of derivatized nanotubes have been inadequate for biological use. Similar to fullerene, biological targeting has not been achieved for nanotube-based therapies, which would significantly increase the probability of producing a nanotube-based therapeutic or diagnostic agent.

### DRAWINGS

Some specific example embodiments of the disclosure may be understood by referring, in part, to the following description and the accompanying drawings.

FIG. 1 shows possible modifications of proteins by N-succinimidyl-3-(2-pyridyldithio)-propionate (SPDP) (a) introduction of a 2-pyridyl disulfide group into a non-thiol protein by aminolysis and (b) introduction of N-hydroxy-succinimide ester structure into a thiol protein by thiol-disulfide exchange.

FIG. 2 shows two possible C₆₀ derivatives designed for conjugation to ZME-018 mAb.

FIG. 3 shows synthesis of N-(3-tert-butylsulfanyl-propyl)malonamic acid ethyl ester for Bingel addition to C₆₀.

FIG. 4 shows a synthesis scheme for the thiol-derivatized C₆₀.

FIG. 5 shows a MALDI TOF mass spectrum of 5a and 5b.

FIG. 6 shows a synthesis scheme for asymmetric amine C₆₀ monoadduct.

FIG. 7 shows a 400 MHz ¹H NMR spectrum of 8 in DMSO-d6

FIG. 8 shows a MALDI TOF mass spectrum of 8 [M⁺]=908.

FIG. 9 shows a synthesis scheme for C₆₀-SPDP Monoadduct.

FIG. 10 shows synthesis of acetate-protected malonodiserinolamide.

FIG. 11 shows a synthesis scheme for water-soluble C₆₀-SPDP

FIG. 12 shows a synthesis scheme for water-soluble C₆₀-Ser.

FIG. 13 shows synthesis of US-tube(Amide), n˜4-5 per nanometer

FIG. 14 shows a) AFM height image of US-tube(Amide) after reduction and b) Z-scan resolution height analysis of US-tube(Amide) after reduction.

FIG. 15 shows a) AFM height image of US-tubes b) AFM height image of 21 from fluorination c) Z-scan resolution height analysis of US-tubes and d) Z-scan resolution height analysis of 21 from fluorination.

FIG. 16 shows TGA of US-tube(Amide) 21, 19, and Mixture.

FIG. 17 shows a) AFM height image of reduced US-tubes b) AFM height image of fluorinated US-tubes c) Z-scan resolution height analysis of reduced US-tubes and d) Z-scan resolution height analysis of fluorinated US-tubes.

FIG. 18 shows a ¹H-¹³C CP-MAS NMR of US-tube(Amide).

FIG. 19 shows a Dipolar dephasing NMR of US-tube(Amide).

FIG. 20 shows the three water-soluble US-tube derivatives.

FIG. 21 shows synthesis of US-tube(Ser).

FIG. 22 shows synthesis of US-tube(PEG).

FIG. 23 shows TGA of US-tube(Ser), 9 and US-tube(PEG)

FIG. 24 shows AFM images of (a) US-tube(Ser) and (b) US-tube(PEG).

FIG. 25 shows Z-scan resolution height analysis of (a) US-tube(Ser) and (b) US-tube(PEG).

FIG. 26 shows 2-iminothiolane conjugation to the ZME-018 mAb.

FIG. 27 shows monoadduct C₆₀-SPDP coupling with the ZME-018 mAb.

FIG. 28 shows a schematic representation showing the formation of the C₆₀-immunoconjugate from C₆₀-SPDP (C₆₀ and antibody figures not to scale).

FIG. 29 shows triplet state decay kinetics of C₆₀-SPDP and C₆₀-SPDP-(ZME-018), as measured at 690 nm following 532 nm excitation.

FIG. 30 shows a) Triplet-Triplet spectrum of C₆₀-SPDP-(ZME-018) immunoconjugate prepared with three different ratios of fullerene to antibody, after chromatographic purification and b) UV absorption spectra of 0.40 μM ZME-018, the C₆₀-SPDP-(ZME-018) immunoconjugate (chromatographically purified), and an unreacted mixture of the two components

FIG. 31 shows UV-vis spectra of the C₆₀-derivatives showing negligible intensity at 595 nm (the Bio-Rad detection wavelength).

FIG. 32 shows UV-vis absorption spectra of a) C₆₀-SPDP-(ZME-018) at 6 μM and C₆₀-SPDP at 30 μM showing that the intensity at 440 nm is not sufficient for concentration determination in the μM range and b) C₆₀-SPDP absorption maximum at 282 nm at 10 μM

FIG. 33 shows ELISA A375m and dead cell testing of C₆₀-ZME-018 immunoconjugates.

FIG. 34 shows TEM images of a) ZME-018 monoclonal antibody b) C₆₀-Ser-(ZME-018) immunoconjugate and c) C₆₀-SPDP-(ZME-018) immunoconjugate. The scale is the same for each frame; scale bar length is 20 nm. The solid curved feature in the image is the lacy carbon grid material.

FIG. 35 shows the nanostructures developed or used to form immunoconjugates with the ZME-018 monoclonal antibody

FIG. 36 shows UV-vis spectrum of Gd@C₆₀(OH)₃₀ and its immunoconjugate.

FIG. 37 shows TEM images of a) Gd—OH-(ZME-018) and b) Gd—COOH-(ZME-018).

FIG. 38 shows ELISA A375m and SK-BR-3 dead cell tests of the Gd@C₆₀-immunoconjugates.

FIG. 39 shows cell internalization of the Gd@C₆₀[C(COOH)₂]₁₀ and Gd@C₆₀(OH)₃₀ immunoconjugates over time.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to compositions and methods related to carbon nanostructures. More particularly, the present disclosure relates to targeted nanostructures and associated methods of use.

In one embodiment, the present disclosure relates to a targeted nanostructure comprising: a Cₙ, a cross-liner, and a targeting agent, wherein Cₙ refers to a fullerene moiety or nanotube comprising n carbon atoms. As used herein, the term “targeting agent” refers to a moiety comprising an antigen-binding site and that is linked to the Cₙ. As used herein, the term “antigen” refers to a chemical compound or a portion of a chemical compound which can be recognized by a specific chemical reaction or a specific physical reaction with another molecule. The antigen-recognition site of an antibody is an exemplary, but non-limiting, antigen-binding site. As used herein, the term “cross linker” refers to anything that is capable of forming links between molecular chains to form a connected molecule.

Cₙ refers to a fullerene moiety comprising n carbon atoms or a nanotube moiety comprising at least n carbon atoms. Examples of suitable Cₙ compounds for use in conjunction with the compositions of the present disclosure, include but are not limited to, buckminsterfullerenes, gadofullerenes, single walled carbon nanotubes (SWNTs), and ultra-short carbon nanotubes (US-tubes). Buckminsterfullerenes, also known as fullerenes or more colloquially, buckyballs, are closed-cage molecules consisting essentially of sp²-hybridized carbons. Fullerenes are the third form of pure carbon, in addition to diamond and graphite. Typically, fullerenes are arranged in hexagons, pentagons, or both. Most known fullerenes have 12 pentagons and varying numbers of hexagons, depending on the size of the molecule. Common fullerenes include C₆₀ and C₇₀ (e.g. n=60 or n=70), although fullerenes comprising up to about 400 carbon atoms are also known. Gadofullerenes (Gd³⁺@C₆₀) refers to gadolinium metal ions enclosed within all-carbon fullerene cages.

SWNTs, also known as single walled tubular fullerenes, are cylindrical molecules consisting essentially of sp² hybridized carbons. In defining the size and conformation of single-walled carbon nanotubes, the system of nomenclature described by Dresselhaus et al., Science of Fullerenes and Carbon Nanotubes, Ch. 19, ibid. will be used. Single walled tubular fullerenes are distinguished from each other by a double index (x,y), where x and y are integers that describe how to cut a single strip of hexagonal graphite such that its edges join seamlessly when the strip is wrapped onto the surface of a cylinder. When x=y, the resultant tube is said to be of the “arm-chair” or (x,x) type, since when the tube is cut perpendicularly to the tube axis, only the sides of the hexagons are exposed and their pattern around the periphery of the tube edge resembles the arm and seat of an arm chair repeated n times. When y=0, the resultant tube is said to be of the “zig-zag” or (x,0) type, since when the tube is cut perpendicular to the tube axis, the edge is a zig zag pattern. Where x≠y and y≠0, the resulting tube has chirality. The electronic properties of the nanotube are dependent on the conformation, for example, arm-chair tubes are metallic and have extremely high electrical conductivity. Other tube types are metallic, semi-metals or semi-conductors, depending on their conformation. Regardless of tube type, all SWNTs have extremely high thermal conductivity and tensile strength. The SWNT may be a cylinder with two open ends, a cylinder with one closed end, or a cylinder with two closed ends. Generally, an end of an SWNT can be closed by a hemifullerene, e.g. a (10,10) carbon nanotube can be closed by a 30-carbon hemifullerene. If the SWNT has one or two open ends, the open ends can have any valences unfilled by carbon-carbon bonds within the single wall carbon nanotube filled by bonds with hydrogen, hydroxyl groups, carboxyl groups, or other groups. SWNTs can also be cut into ultra-short pieces, thereby forming US-tubes. As used herein, the term “US-tubes” refers to ultra short carbon nanotubes with lengths from about 20 nm to about 100 nm.

The Cₙ can be substituted or unsubstituted. By “substituted” it is meant that a group of one or more atoms is covalently linked to one or more atoms of the Cₙ. Generally, in situ Bingel chemistry may be used to substitute the Cₙ with appropriate groups to form the targeted nanostructures of the present disclosure. Examples of groups suitable for use include, but are not limited to, malonate groups, serinol malonates, groups derived from malonates, serinol groups, carboxylic acid, polyethyleneglycol (PEG), and the like. In one embodiment, the Cₙ is substituted with one or more water-solubilizing groups. Water-solubilizing groups are polar groups (that is, groups having a net dipole moment) that render the generally hydrophobic fullerene core soluble in water. The addition of such groups allow for greater biocompatibility of the Cₙ. Generally, the Cₙ may contain from 1 to 4 addends. The Cₙ can be substituted with any water solubilizing group to allow for sufficient water solubility and biocompatibility, but the spectroscopic properties of the Cₙ should not be compromised. In certain embodiments, the Cₙ may be further substituted with either a thiol (—SH) or an amine (—NH₂) group to aid in the coupling of the cross linker to the Cₙ moiety.

The cross linker may comprise any group capable of linking the Cₙ to the targeting agent. In certain embodiments, the cross linker may be covalently bound to the portion of the targeting agent containing the antigen bonding site and capable of associating with the Cₙ. In other embodiments, the cross linker may be physically associated with the Cₙ. Examples of cross linkers suitable for use in conjunction with the compositions of the present disclosure include but are not limited to, N-succinimidyl-3-(2-pyridyldithio)-propionate (SPDP) and serinol. In certain embodiments, the cross linker may be attached directly to an amine substituted Cₙ moiety. In other embodiments, the cross linker may be used to derivatize the targeting agent and attached to a thiol substituted Cₙ moiety.

The targeting agent used in conjunction with the present disclosure may be attached to the fullerene molecule by the cross linker. The targeting agent may be a protein, an antibody, or a portion of an antibody, such as a glycogen IIa/IIB receptor antibody, Von Willebrand's factor antibody, an antitumor antibody, hepatic cellular antibody, a white blood cell antibody, and antifibrin. Examples of moieties comprising antigen-binding sites that may be used as targeting agents include, but are not limited to, monoclonal antibodies, polyclonal antibodies, Fab fragments of monoclonal antibodies, Fab fragments of polyclonal antibodies, Fab₂ fragments of monoclonal antibodies, and Fab₂ fragments of polyclonal antibodies, among others. Single chain or multiple chain antigen-recognition sites can be used. Multiple chain antigen-recognition sites can be fused, joined by a linker, or unfused and unlinked.

The targeting agent can be selected from any known class of antibodies. Known classes of antibodies include, but are not necessarily limited to, IgG, IgM, IgA, IgD, and IgE. The various classes also can have subclasses. For example, known subclasses of the IgG class include, but are not necessarily limited to, IgG1, IgG2, IgG3, and IgG4. Other classes have subclasses that are routinely known by one of ordinary skill in the art.

Similarly, the targeting agent can be derived from any species. “Derived from,” in this context, can mean either prepared and extracted in vivo from an individual member of a species, or prepared by known biotechnological techniques from a nucleic acid molecule encoding, in whole or part, an antibody peptide comprising invariant regions which are substantially identical to antibodies prepared in vivo from an individual member of the species or an antibody peptide recognized by antisera specifically raised against antibodies from the species. Exemplary species include, but are not limited to, human, chimpanzee, baboon, other primate, mouse, rat, goat, sheep, and rabbit, among others known in the art. In certain embodiments, the targeting agent may be chimeric, i.e., comprises a plurality of portions, wherein each portion is derived from a different species. A chimeric antibody, wherein one of the portions is derived from human, can be considered a humanized antibody.

Targeting agents are available that recognize antigens associated with a wide variety of cell types, tissues, and organs, and a wide variety of medical conditions, in a wide variety of mammalian species. Examples of medical conditions include, but are not limited to, cancers, such as lung cancer, oral cancer, skin cancer, stomach cancer, colon cancer, nervous system cancer, leukemia, breast cancer, cervical cancer, prostate cancer, and testicular cancer; arthritis; infections, such as bacterial, viral, fungal, or other microbial infections; and disorders of the skin, the eye, the vascular system, or other cell types, tissues, or organs; among others.

Examples of targeting agents include, but are not limited to, those derived from antibodies against anthrax or other bacteria, antibodies against the spores of anthrax or other bacteria, antibodies against vascular endothelial growth factor receptor (VEGF-r) (available from Imclone, New York, N.Y.), antibodies against epidermal growth factor receptor (EGF-r) (available from Abgenix, Fremont, Calif.), antibodies against polypeptides associated with lung cancers (available from Corixa Corporation, Seattle, Wash.), antibodies against human tumor necrosis factor alpha (hTNF-.alpha.) (available from BASF A.G., Ludwigshafen, Germany), among others known in the art.

Suitable targeting agents can be prepared by various techniques that are known in the art. These techniques include, but are not limited to, the immunological technique described by Kohler and Milstein in Nature 256, 495-497 (1975) and Campbell in “Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1985); as well as by the recombinant DNA techniques described by Huse et al in Science 246, 1275-1281 (1989); among other techniques known to one of ordinary skill in the art.

In addition to the listed antibodies, the targeting agent can be constructed to recognize a target antigen associated with a solid tumor. For example, the targeting agent can be constructed to recognize HER2/neu, MUC-1, HMFG1, or EGFr, associated with breast tumors; MMP-9, HER2/neu, or NCAM, associated with lung tumors; HER2 or 171A, associated with colon tumors; gp240, gangliosides, or integrins, associated with melanomas; HER2 or CA-125, associated with ovarian tumors; or EGFr or tenascin, associated with brain tumors. In certain embodiments, the targeting agent may comprise ZME-018 monocolonal antibody against gp240 in melanoma cells.

The targeted nanostructures of the present disclosure may further comprise a contrast agent. As used herein, the term “contrast agent” refers to any agent which is detectable by any means. Examples of contrast agents, include but are not limited to, MRI contrast agents (e.g. magnetic metal particles), computed tomography (CT) contrast agents (e.g. hyperpolarized gas), X-ray contrast agents, nucleosan contrast agents, and ultrasonic contrast agents, among others. The contrast agents of the present disclosure are generally sequestered within the carbon nanostructures. Generally all or a portion of the carbon nanostructure may be loaded with contrast agent. Specific examples of some suitable contract agents may include magnetic metallic particles, such as Gd³⁺, I₂, and any iodine moiety. Accordingly, the targeted nanostructures of the present disclosure may comprise an iodine loaded fullerene, an iodine loaded nanotube, a gadofullerene, or a gadolinium loaded nanotube.

In certain embodiments, the targeted nanostructures of the present disclosure may be imaged using imaging techniques known in the art, such as CT, MRI, and the like, depending on the particular contrast agent chosen. For example, the target nanostructures may be administered to a subject (e.g., a human or animal) or used in an assay and allowed to interact with an antigen. Subsequently, the targeted nanostructures may be imaged.

To facilitate a better understanding of the present invention, the following examples of specific embodiments are given. In no way should the following examples be read to limit or define the entire scope of the invention.

### EXAMPLES

All reagents used were reagent grade or better. Anhydrous material purification was performed under N₂ or Ar (Trigas, purified) atmosphere. Further purification of inert gases was performed in a schlenk line containing R3-11 catalyst (Chemical Dynamics Corp.) on vermiculite and Drierite (CaSO₄). For anaerobic reactions, the solvents were degassed with N₂ or Ar. Desiccators contained Drierite desiccant. Solvent purification procedures were performed according to literature precedent.

The following reagents were used as received: C₆₀ (99.5+% pure, MER Corp.), tetrahydro-1,3-thiazine-2-thione (Aldrich), CS₂ (Aldrich), tert-butanol (Aldrich), P2O5 (Fisher), NaHCO₃ (Fisher), ethyl malonyl chloride (Aldrich), NaCl (Fisher), MgSO₄ (Fisher), CBr₄ (Aldrich), DBU (Aldrich), tert-butyl N-(3-hydroxypropyl)-carbamate (Aldrich), TFA (Acros), NaOH (Fisher), conc. HCl (Fisher), SPDP (Pierce), 2-amino-1,3-propanediol (Aldrich), CuSO₄ (Baker), Na₂CO₃ (Fisher), diethyl malonate (Aldrich), 1% F2 gas in He (Air Products), HiPco Single-walled carbon nanotubes (SWNTs) (Carbon Nanotechnologies Inc.), Na metal (Aldrich), K metal (Fisher), malonyl dichloride (Aldrich), NaH (Acros), oxalyl chloride (Aldrich), PEG (Aldrich) H4EDTA (Aldrich), CaSO₄ (Drierite), CaH₂ (Acros), 2-iminothiolane (Pierce), iodoacetamide (Aldrich), Na₃PO₄ (Fisher), urea (Pierce).

The following reagents were purified as described: TEA (Acros) was refluxed and distilled from CaH₂ and Ac₂O (Fisher) was distilled.

The following solvents were used as received: petroleum ether (Fisher), acetone (Fisher) and DI water from the laboratory DI faucet.

The following solvents were purified as described: toluene (Fisher) was distilled over Na with a benzophenone indicator, methylene chloride (Fisher) was pre-dried with CaCl₂ and distilled over P₂O₅, EtOAc (Fisher) was distilled from MgSO₄, pyridine (Fisher) was dried with KOH with distillation over molecular sieves and solid KOH, chloroform (Fisher) was distilled over CaCl₂, MeOH (Fisher) was distilled, hexanes (Fisher) were dried with CaCl₂ and distilled over molecular sieves, EtOH (Fisher) was distilled over CaSO₄, THF (Fisher) was distilled from K/Na.

Column chromatography was performed with 70-230 mesh silica gel powder, which was slurry-packed using toluene as the solvent. ZME-018 immunoconjugates were purified with a G-25 sephadex size-exclusion chromatography, after which Bio-Rad protein assays determined the concentration of ZME-018 in the immunoconjugate solution. Enzyme-linked immunosorbent assay (ELISA) was utilized to establish the IC50 value for each immunoconjugate. An ELX 800 UV-vis spectrometer from Bio-Tek Instrument was used to analyze the Bio-Rad assay and ELISA plates at 595 nm. Thin layer chromatography was carried out using silica gel 60, F-254 flexible TLC plates.

High-performance liquid chromatography (HPLC) purification was accomplished on a Hitachi L-6200A Intelligent Pump HPLC system with a Hitachi Model L-3000 UV-vis photodiode array detector using an econosil silica 10μ column (Alltech).

The cation-exchange resin (Bio-Rad) AG 50W-X2 (H+ form) removed cations from the serinol adducts of fullerene. Before use, the resin was washed extensively with DI water.

Nuclear magnetic resonance (NMR) solvents were used as received from Cambridge Isotope Laboratories. NMR spectra were obtained on a Bruker 400 MHz spectrometer. Solid-state ¹³C NMR spectra were obtained on a Bruker AVANCE-200 NMR spectrometer (50.3 MHz ¹³C, 200.1 MHz ¹H). A Perkin Elmer Paragon 1000 PC spectrometer collected FT-IR spectra. UV-Vis spectroscopy was performed on a Cary 4 spectrometer with a 1.0 mm quartz cell containing 500 μl of sample in water. The water solubilities and n-octanol/water partition coefficients of the C₆₀ and nanotube materials were determined by UV-vis spectroscopy at 25° C. by the method of Leo.

Mass Spectra were obtained on a Finnigan Mat 95 mass spectrometer or a Bruker Biflex III MALDI-TOF mass spectrometer. For the MALDI spectra, an elemental sulfur matrix was added to analyte and deposited on the sample plate.

Triplet-triplet absorption measurements and triplet-state decay kinetics were determined after excitation with a 532 nm pulse from a small Q-switched Nd:Yag laser. The samples were dissolved in water and freeze-pumped-thaw degassed three times to remove oxygen.

Thermal Gravimetric Analysis (TGA) was carried out using a SEIKO 1 TG/DTA 200 instrument with an Al pan under argon. The temperature was ramped 10° C./min.

Transmission electron microscopy (TEM) images were captured with a single drop of nanomaterial deposited on a 300 mesh copper grid, Lacey Carbon Type-A support film, manufactured by Ted Pella, Inc. The sample was allowed to air-dry for 5 min under ambient conditions before imaging. A JEOL 2010 model TEM, operating at 100 keV imaged samples at 30,000× and 80,000× magnification.

Atomic force microscopy (AFM) was obtained using samples which were spin coated on a mica wafer after dispersion and sonication in THF, followed by AFM analysis using tapping mode on a DI Nanoscript 3A instrument.

The concentration of gadolinium in the Gd@C₆₀[C(COOH)₂]₁₀ and Gd@C₆₀(OH)₃₀ samples and immunoconjugates were determined using ICP-AE with a Varian Vista Pro Simultaneous Axial Inductively Coupled Atomic Emission Spectrometer with an atomic emission CCD detector. A calibration curve was obtained using 0.1, 1, 2, 4, 8 and 16 ppm Gd³⁺ standard and sample concentrations were collected three times in replicate with a standard deviation of <2%. The Gd³⁺ concentration for the cell internalization studies were acquired with a Perkin-Elmer Elan 900 inductively coupled plasma-mass spectrometer (ICP-MS). A calibration curve was produced from 0.1, 0.5, 1, 2, 4, 8 and 16 ppb Gd³⁺ standards and sample concentrations collected three times in replicate with standard deviation of <2%. The Gd@C₆₀[C(COOH)₂]₁₀ and Gd@C₆₀(OH)₃₀ samples were graciously donated by TDA Research Inc. of Wheat Ridge, Colo.

Nanotube functionalization was characterized by elemental analysis using a PHI Quantera X-ray photoelectron spectrometer (XPS). A Monel flow apparatus using a gaseous mixture of 1% F₂ in He was used to fluorinate the SWNTs, which were then pyrolyzed at 1000° C. in a tube furnace to produce US-tubes. The HiPco SWNTs were obtained from Carbon Nanotechnolgies, Inc. of Houston, Tex.

### Example 1

### Fullerene Chemistry

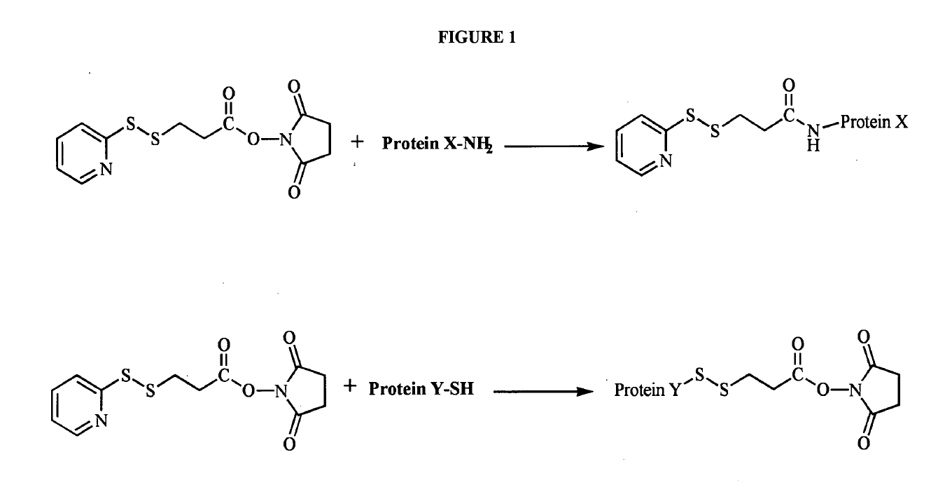

SPDP is a heterobifunctional cross linker, which can undergo aminolysis with its N-hydroxysuccinimide ester (a in FIG. 1) or disulfide exchange with 2-pyridyldisulfide (b in FIG. 1). Recently, SPDP has linked human IgM mAb 16-88 to cobra venom factor, mAb 138H11 to the DNA-cleaving enediyne, calicheamicin and a fifth generation polyamidoamine (starburst) dendrimer to oligosaccharide moieties away from the antigen binding site of the chimeric mAb, cetuximab. It is feasible to derivatize C₆₀ with either a thiol (—SH) or an amine (—NH₂), with subsequent coupling with a SPDP derivatized antibody for the thiol derivatized C₆₀ or direct attachment of SPDP to the amine derivatized C₆₀. The advantages and disadvantages of each C₆₀-derivative were compared in order to ascertain which derivative was more conducive for conjugation to the ZME-018 mAb.

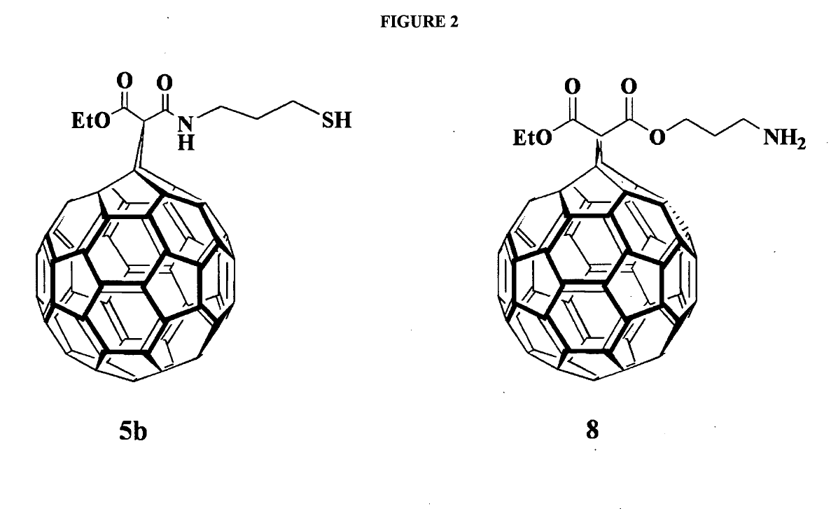

Initial coupling of SPDP to proteins normally occurs via aminolysis (a in FIG. 1), followed by attachment of a thiol-containing compound through new disulfide bond formation with the SPDP-conjugated protein (b in FIG. 1). Therefore, attempts to synthesize a C₆₀-thiol derivative (6b in FIG. 2) for conjugation to the ZME-018 mAb were first explored.

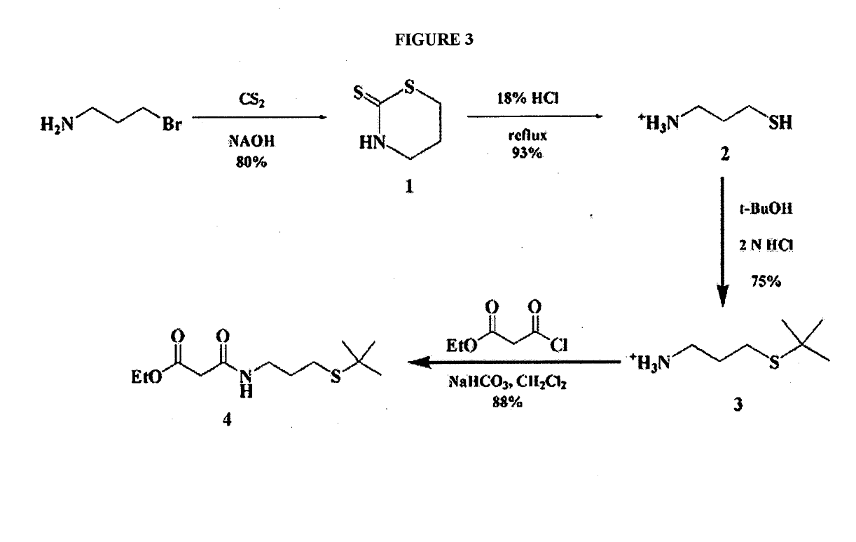

First, N-(3-tert-butylsulfanyl-propyl)malonamic acid ethyl ester (4 in FIG. 3) was prepared for attachment to C₆₀. The synthesis began by reacting 3-bromopropylammine with carbon disulfide to form cyclic tetrahydro-1,3-thiazine-2-thione, 1. Acid hydrolysis of 1 with 18% hydrochloric acid and heat produced 3-amino-propane-1-thiol, 2. The thiol functionality was then protected with tert-butyl to form 3. Finally, nucleophilic substitution of ethyl malonyl chloride with the primary amine from 3 gave the desired malonate, 4, for use in Bingel addition to C₆₀.

### Preparation of Tetrahydro-1,3-thiazine-2-thione (1)

In a 100 mL round bottom flask, 10.0 g (0.046 moles) of 3-bromopropylamine hydrobromide was chilled on ice. While stirring, 3 molar equivalents, 10.4 g (0.14 moles) of CS₂ was added. In a separate round bottom flask, 4.0 g of NaOH (0.10 moles) was dissolved in 25 mL of ice cold DI H₂O. The two solutions were combined and stirred overnight at 0° C. The solid product was collected by vacuum filtration and washed three times with DI H₂O. The crude solid was purified by recrystallization in EtOH to give 4.5 g (0.034 moles) of pure 1 as a white powder; yield 74%. mp 114-116° C.; ¹H NMR (400 MHz, CDCl₃) δ (ppm) 2.20 (p, 2H, CH₂), 3.00 (t, 2H, CH₂), 3.48 (t, 2H, CH₂), 8.63 (bs, 1H, NH); ¹³C NMR (400 MHz, CDCl₃) δ (ppm) 20.91 (1C, CH₂), 30.41 (1C, CH₂), 44.80 (1C, CH₂), 195.28 (1C, C═O); FT-IR (KBr) ν (cm⁻¹) 3442, 1647 (N—H), 2361 (C—S), 1547 (N—C═S); EI-MS calculated for C₄H₈NS₂ (M⁺) 134.0, found 134.0.

### Preparation of 3-Amino-propane-1-thiol (2)

10.0 g of 1 (0.075 moles) was dissolved in 75 mL of 18% HCl to generate a bright yellow solution, which was refluxed until the bright yellow color converted to a clear liquid. The solvent was then removed under reduced pressure to yield an impure white solid coated with a clear oily residue. The crude product was purified in a scintillation vial by placing it under high vacuum overnight. This caused the oily residue to evaporate, leaving 9.1 g (0.072 moles) of pure 2 as a white solid; yield 95%. ¹H NMR (400 MHz, D₂O) δ (ppm) 1.98 (p, 2H, CH₂), 2.60 (t, 2H, CH₂), 3.10 (t, 2H, CH₂); ¹³C NMR (400 MHz, D₂O) δ (ppm) 20.88 (1C, CH₂), 30.88 (1C, CH₂), 38.38 (1C, CH₂); EI-MS calculated for C₃H₉NS (M+) 91.0, found 91.0.

### Preparation of 3-tert-Butylsulfanylpropylamine (3)

2.0 g (0.016 moles) of 2 was combined with 1.5 g (0.020 moles) of tert-butanol and refluxed in 7.0 mL (0.014 moles) of 2 N HCl for 12 hrs. The HCl was then removed under reduced pressure to leave a white solid which was then retreated to 3.5 mL of 2 N HCl and tert-butanol and condensed an additional 10 hr. After removal of HCl a crude solid remained that was purified by recrystallization from toluene to give a white solid. This solid was then placed in a scintillation vial and dried overnight with P₂O₅ in a drying pistol, giving 2.3 g (0.015 moles) of pure 3; yield 96%. ¹H NMR (400 MHz, D₂O) δ (ppm) 1.30 (s, 9H, CH₃), 1.95 (p, 2H, CH₂), 2.67 (t, 2H, CH₂), 3.10 (t, 2H, CH₂); ¹³C NMR (400 MHz, D₂O) δ (ppm) 24.73 (1C, CH₂), 27.45 (1C, CH₂), 30.24 (3C, CH₃), 39.06 (1C, C—S), 43.15 (1C, CH₂); EI-MS calculated for C₇H₁₈NS (M⁺) 148.1, found 148.0.

### Preparation of N-(3-tert-butylsulfanyl-propyl)-malonamic acid ethyl ester (4)

0.9 g (0.006 moles) of 3 was dissolved in 30 mL of anhydrous CH₂Cl₂ on ice and combined with 30 mL of ice-cold saturated sodium bicarbonate. Drop-wise addition of 1.3 mL (0.01 moles) of ethyl malonyl chloride in 6 mL of CH₂Cl₂ initiated the nucleophilic substitution reaction. The solution was stirred on ice for 20 min, followed by stirring at room temperature overnight. DI H₂O was added to quench the reaction and the crude product was obtained by extraction with EtOAc. The aqueous portion was washed three times with EtOAc, which was added to the organic layer. Further washing of the organic layer was accomplished with water (three times) and brine (three times), with subsequent drying over MgSO₄. MgSO₄ was removed by gravity filtration and EtOAc removed under reduced pressure to give impure 4. The crude product was purified using column chromatography with chloroform as the eluant on silica gel, giving 1.2 g (0.0046 moles) of 4 as a golden viscous liquid; yield 92%. ¹H NMR (400 MHz, CDCl₃) δ (ppm) 1.25 (t, 3H, CH₃), 1.28 (s, 9H, CH₃), 1.72 (p, 2H, CH₂), 2.49 (t, 2H, CH₂), 3.23 (s, 2H, CH₂), 3.28 (q, 2H, CH₂), 4.14 (q, 2H, CH₂); ¹³C NMR (400 MHz, CDCl₃) δ (ppm) 14.43 (1C, CH₃), 26.00 (1C, CH₂), 29.84 (1C, CH₂), 31.29 (3C, CH₃), 39.24 (1C, CH₂), 41.66 (1C, C—S), 42.46 (1C, CH₂), 61.90 (1C, CH₂), 165.48 (1C, C═O), 169.87 (1C, C═O); EI-MS calculated for C₁₂H₂₃O₃NS (M⁺) 261.0, found 261.1.

### Preparation of the C₆₀-Amine Derivative

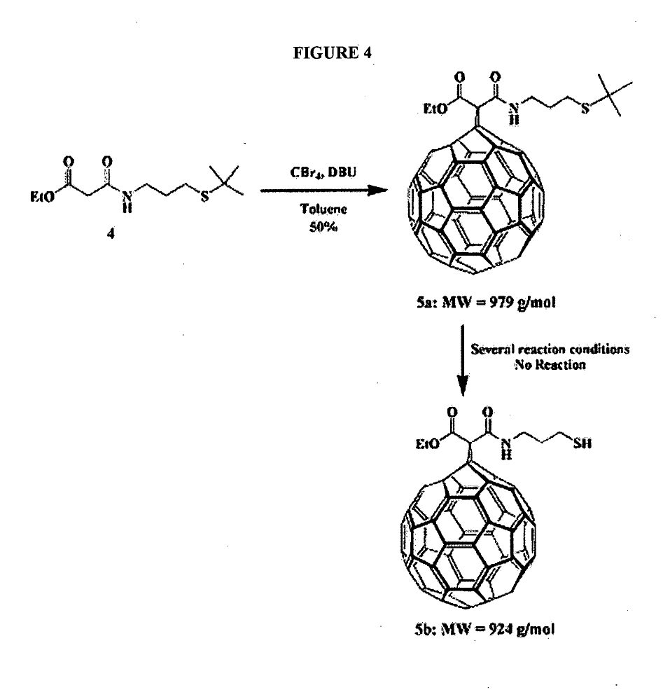

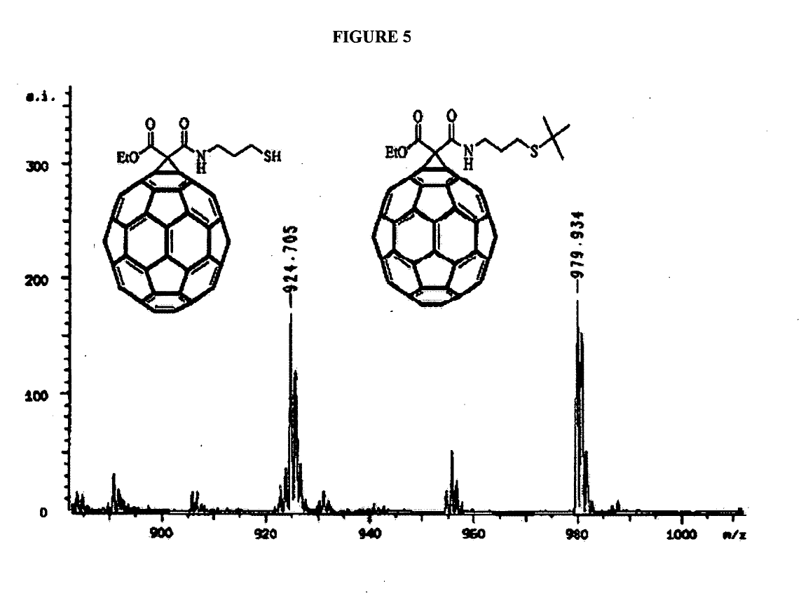

Preparation of the C₆₀-thiol derivative 5b (FIG. 4) proved problematic. After Bingel reaction of 4 to C₆₀, MALDI TOF-MS of 5 (FIG. 5) contained the desired molecular weight of the protected thiol malonate adduct (5a, M⁺=980). However, the free thiol molecular ion peak (5b, M⁺=925) was also evident. Unfortunately, it was not possible to determine if this was an ionization fragment or an actual molecular ion peak of 5b. Several attempts to remove the tert-butyl protecting group on 5a using various reaction conditions were unsuccessful. Another difficulty was the inability to obtain a ¹H NMR spectrum due to peak broadening, which is characteristic of paramagnetic behavior that had been demonstrated by several amide Bingel products.⁸³ Therefore, an alternative C₆₀-amine derivative, 8, was prepared for coupling to the ZME-018 mAb. The C₆₀ derivative, 8, contains an amine arm that is capable of attaching to SPDP for coupling to thiol-derivatized antibodies. A drawback to this method is that the antibody must first be derivatized with 2-iminothiolane, forming a free-thiol that can then displace the disulfide on the SPDP attached to C₆₀. This increased the steps in antibody preparation, but allowed for a more useful functionalization of C₆₀.

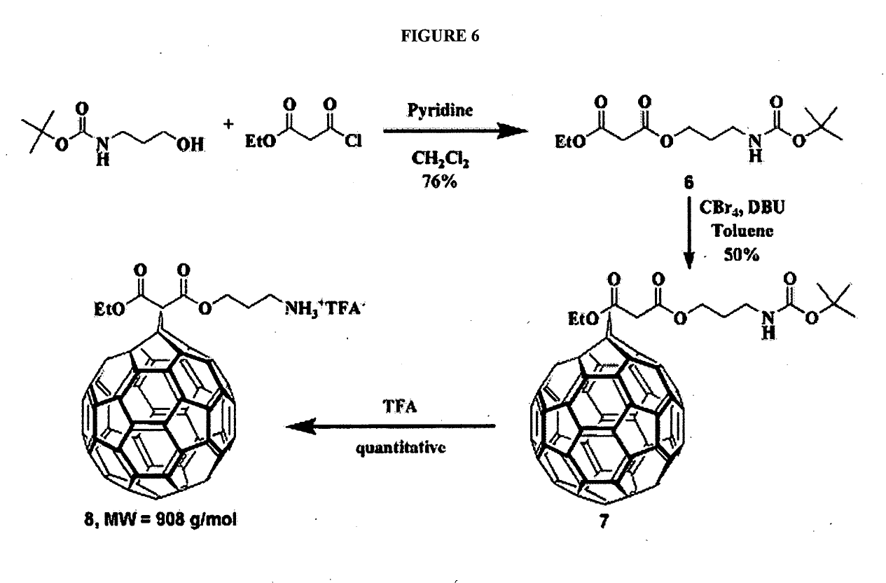

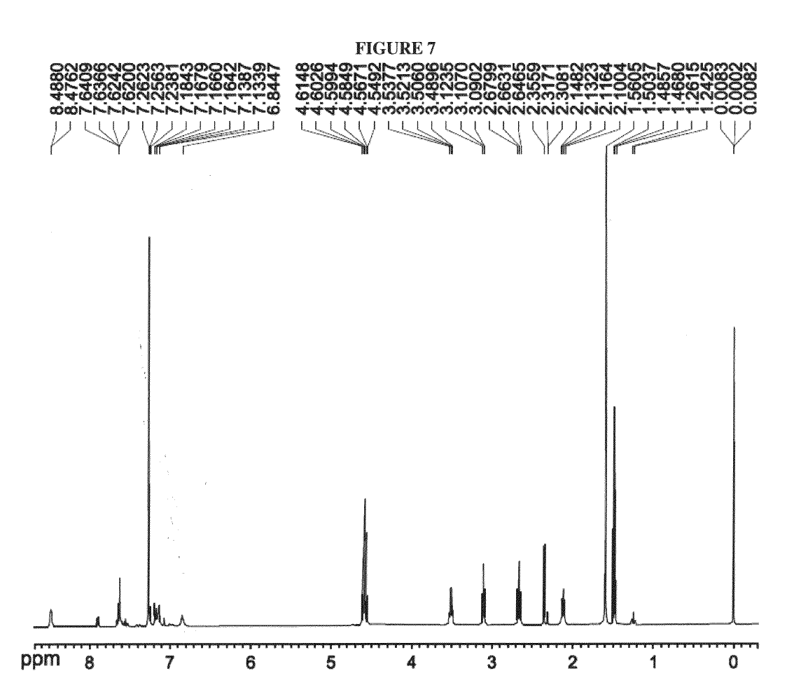

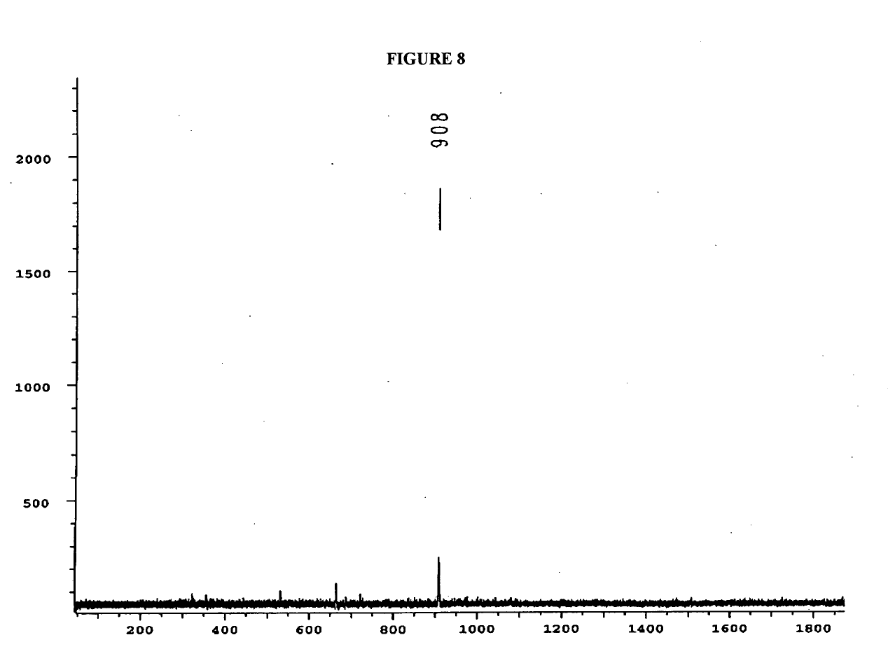

Synthesis of 8 was accomplished as shown in FIG. 6. First, nucleophilic substitution of ethyl malonyl chloride with tert-butyl-N-(3-hydroxypropyl)carbamate formed the asymmetric malonate, 6. For the C₆₀-antibody coupling an asymmetric malonate was desired for several reasons: (1) to allow for future fluorescent tagging through the non-conjugated malonate arm, (2) to allow for a single SPDP molecule attachment to the antibody per C₆₀ moiety, and (3) to allow for future drug attachment to the non-conjugated malonate arm in order to facilitate targeted drug delivery. The asymmetric malonate, 6, was attached to C₆₀ using in situ Bingel conditions, giving the protected amine C₆₀, 7. Deprotection of the tert-butoxy protecting group with TFA liberated the primary amine, forming the desired C₆₀ derivative, 8, which was characterized using NMR (FIG. 7) and MALDI-TOF MS (FIG. 8).

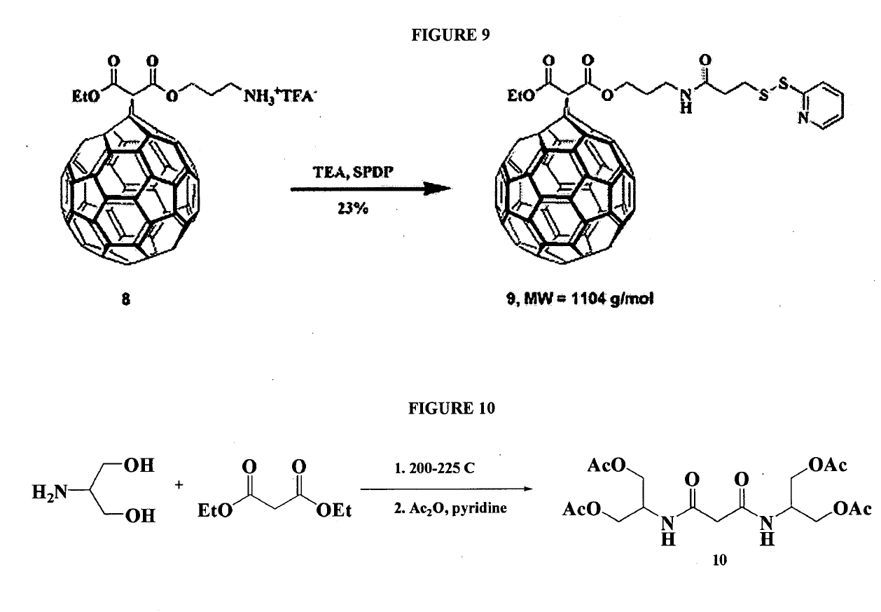

A C₆₀-SPDP monoadduct (9 in FIG. 9) was then prepared via aminolysis to test the feasibility of attaching the cross-linker, SPDP to C₆₀. TEA was added slowly to 8, followed by the addition of SPDP to form the amide linkage to C₆₀, 9, with release of N-hydroxysuccinimide. The following illustrates the preparation of the (C₆₀) derivative.

### Preparation of Asymmetric-Protected Thiol Fullerene (C₆₀) Derivative (5)

Using Bingel chemistry, 500 mg (0.69 mmol) of C₆₀ was dissolved in 500 mL of anhydrous toluene in a 1000 L round bottom flask. 45.3 mg (0.17 mmol) of 4 was then added to the reaction flask followed by 56.4 mg (0.17 mmol) of CBr₄ and drop-wise addition of 52.4 mg (0.34 mmol) of DBU. Stirring continued for 1 hr, and the toluene was removed under reduced pressure. Unreacted C₆₀ was removed on a silica gel column using toluene as the eluant. A 10:1 toluene/EtOAc eluant was then used to obtain the pure C₆₀ monoadduct. The solvent was removed under reduced pressure to give 71.6 mg (0.073 mmol) of 5 as a red solid; yield 43%. The ¹H NMR spectrum could not be obtained due to broadening of the spectral signals. This is likely due to the product being somewhat paramagnetic. MALDI-MS calculated for C₇₂H₂₁O₃NS (M⁺) 979.0, found 979.9 (FIG. 5). Removal of the tert-butyl protecting group to form the free thiol derivative of fullerene (5b) proved fruitless.

### Preparation of Malonic Acid 3-Tert-Butoxycarbonylamino-Propyl Ester Ethyl Ester (6)

According to published procedure, 2.5 g (0.014 moles) of tert-butyl-N-(3-hydroxypropyl)carbamate and 1.5 mL (0.019 moles) of pyridine were combined in 100 mL of anhydrous CH₂Cl₂. The solution was cooled in an ice bath, during which 2.0 g (0.013 moles) of ethyl malonyl chloride was added drop-wise to the reaction flask under nitrogen. The mixture was stirred at room temperature for 12 hr. followed by quenching of the reaction with DI H₂O. Extraction was performed using CH₂Cl₂ with subsequent washing of the organic layer three times with DI H₂O. The CH₂Cl₂ was removed under reduced pressure, and the crude product purified on a silica gel column using a 1:1 hexanes/EtOAc eluant to give 2.9 g (0.0098 moles) of pure 6 as a pale yellow oil; yield 76%. ¹H NMR (400 MHz, CDCl₃) δ (ppm) 1.28 (t, 3H, CH₃), 1.43 (s, 9H, CH₃), 1.85 (p, 2H, CH₂), 3.20 (m, 2H, CH₂), 3.39 (s, 2H, CH₂), 4.18-4.28 (m, 4H, CH₂), 5.30 (bt, 1H, NH); ¹³C NMR (400 MHz, CDCl₃) δ (ppm) 14.06 (1C, CH₃), 28.70 (3C, CH₃), 28.94 (1C, CH₂), 37.23 (1C, CH₂), 41.50 (1C, CH₂), 61.42 (1C, CH₂), 62.88 (1C, CH₂), 78.88 (1C, O—C), 156.10 (1C, C═O), 166.61 (1C, C═O), 166.73 (1C, C═O) EI-MS calculated for C₁₃H₂₃O₆N (M⁺) 290.2, found 290.4.

### Preparation of Asymmetric-Protected Amine Fullerene (C₆₀) Derivative (7)

500 mg (0.69 mmol) of C₆₀ was dissolved in 700 mL of toluene, followed by sequential addition of 100 mg (0.34 mmol) 6, 120 mg (0.36 mmol) of CBr₄ and 105 mg (0.69 mmol) of DBU with stirring at room temperature for 1 hr. Toluene was removed under reduced pressure, and the C₆₀ monoadduct purified with column chromatography on a silica gel column using toluene as eluant to remove non-reacted C₆₀. This was followed by a 10:1 toluene/EtOAc eluant to give 170 mg (0.17 mmol) of pure 7 as a reddish-brown solid; yield 50%. ¹H NMR (400 MHz, CDCl₃) δ (ppm) 1.46-1.54 (m, 12H, CH₃), 2.06 (p, 2H, CH₂), 3.38 (m, 2H, CH₂), 4.52-4.60 (m, 4H, CH₂), 4.79 (bt, 1H, NH); ¹³C NMR (400 MHz, CDCl₃) δ (ppm) 14.30 (1C, CH₃), 28.43 (3C, CH₃), 29.18 (1C, CH₂), 37.31 (1C, CH₂), 52.10 (bridgehead C), 63.57 (1C, CH₂), 64.86 (1C, CH₂), 71.52 (C₆₀ sp³ C) 79.47 (1C, O—C), 138.84, 139.18, 140.99, 141.89 142.21, 143.04, 143.90, 144.64, 144.66, 144.70, 144.91, 145.14, 145.16, 145.20, 145.29 (C₆₀ sp² C), 155.94 (1C, C═O), 163.58 (1C, C═O), 163.79 (1C, C═O); MALDI-MS calculated for C₇₃H₂₁O₆N (M⁺) 1008, found 1007.

### Preparation of Asymmetric-Amine Fullerene (C₆₀) Derivative (8)

170 mg (0.17 mmol) of 7 was dissolved in 100 mL of a 1:1 CH₂Cl₂/TFA solution and stirred for 30 min. The solvents were evaporated off under reduced pressure giving 155 mg (0.17 mmol) 8 as a reddish-brown solid, yield 100%. ¹H NMR (400 MHz, DMSO-d6) δ (ppm) 1.40 (t, 3H, CH₃), 2.09 (p, 2H, CH₂), 2.95 (m, 2H, CH₂), 4.51-4.57 (m, 4H, CH₂), 7.89 (s, 3H, NH₃⁺); ¹³C NMR (400 MHz, DMSO-d6.) δ (ppm) 14.95 (1C, CH₃), 27.15 (1C, CH₂), 36.87 (1C, CH₂), 52.97 (bridgehead C), 64.55 (1C, CH₂), 65.45 (1C, CH₂), 72.17 (C₆₀ sp³ C), 139.24, 141.35, 142.22, 142.23 142.57, 143.37, 143.42, 144.24, 144.96, 145.04, 145.07, 145.22, 145.46, 145.50, 145.56 (C₆₀ sp² C), 163.48 (1C, C═O), 163.52 (1C, C═O); MALDI-MS calculated for C₆₈H₁₄O₄N (M⁺) 908, found 908.

### Preparation of Asymmetric-SPDP Fullerene (C₆₀) Derivative (9)

TEA was added drop-wise to 150 mg (0.165 mmol) of 8 in 20 mL of anhydrous CH₃Cl until the solid completely dissolved, followed by addition of 50 mg (0.160 mmol) of SPDP at room temperature with stirring overnight. The product was purified with column chromatography on silica gel using a 1:1 ratio of toluene/EtOAc as the eluant. Additional purification was performed using HPLC with a 15:1 ratio of toluene/MeOH eluant to give 40 mg (0.036 mmol) of 9 as a reddish-brown solid; yield 23%. ¹H NMR (400 MHz, CDCl₃) δ (ppm) 1.48 (s, 3H, CH₃), 2.10 (p, 2H, CH₂), 2.66 (t, 2H, CH₂), 3.10 (t, 2H, CH₂), 3.50 (q, 2H, CH₂), 4.54-4.61 (m, 4H, CH₂), 6.84 (bt, 1H, NH), 7.16 (m, 1H, ArH), 7.62 (m, 2H, ArH), 8.48 (d, 1H, ArH); ¹³C NMR (400 MHz, CDCl₃) δ (ppm) 14.52 (1C, CH₃), 29.01 (1C, CH₂), 35.33 (1C, CH₂), 36.08 (1C, CH₂), 36.68 (1C, CH₂), 52.30 (bridgehead C), 63.87 (1C, CH₂), 65.23 (1C, CH₂), 71.70 (C₆₀ sp³ C), 120.69 (1C, ArC), 121.37 (1C, ArC), 125.51 (1C, ArC), 137.31, 139.08, 139.33, 142.08, 142.10, 142.42, 143.23, 143.26, 144.10, 144.86, 144.91, 145.12, 145.33, 145.40, 145.41, 145.50 (C₆₀ sp² C), 149.81 (1C, ArC), 163.88 (C═O), 163.89 (C═O), 171.35 (C═O); MALDI-TOF MS calculated for C₇₆H₂₀O₅N₂S₂: 1104; found: 1105.

### Example 2

### Biocompatible C₆₀ Derivatives

For successful coupling of C₆₀ to ZME-018 to occur, the C₆₀-SPDP derivative must display sufficient water solubility. Previously, attachment of serinol malonates, which consist of four hydroxyl water-solubilizing groups, have shown astounding C₆₀ water-solubilizing abilities. In fact, these malonates are the most efficient C₆₀ water-solubilizing adducts to date.³⁹ Thus, attaching multiple serinol moieties to the exterior of C₆₀⁸⁵ was used to obtain high water solubility for the C₆₀-SPDP derivative, while retaining the ability to functionalize so that coupling to ZME-018 occurred in a facile manner.

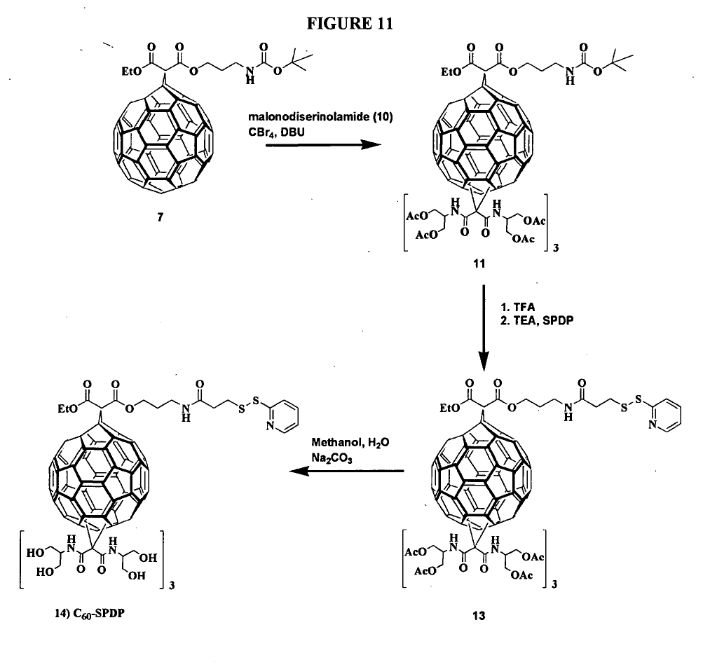

C₆₀-SPDP was made biocompatible by derivatization with 10 (synthesis shown in FIG. 10), followed by subsequent removal of the acetate protecting groups. First, diethyl malonate was condensed with serinol, with concomitant protection of the hydroxyl functional groups with acetate to give 10. As before, in situ Bingel addition was utilized to attach an average of three adducts of 10 to 7 (using a 5:1 ratio of 10:7) to form 11 (FIG. 11).

Biocompatible C₆₀-SPDP 14 was obtained in three steps from 11. The tert-butoxy protecting group was removed with TFA to give the primary amine. Then aminolysis of SPDP with the primary amine of 11 was accomplished, yielding 13. Finally, the acetate protecting groups are cleaved, liberating the water-solubilizing hydroxyl functionalities to give biocompatible C₆₀-SPDP, 14. Attachment of SPDP to C₆₀, before the removal of acetate protecting groups, is vital for the successful preparation of 14.

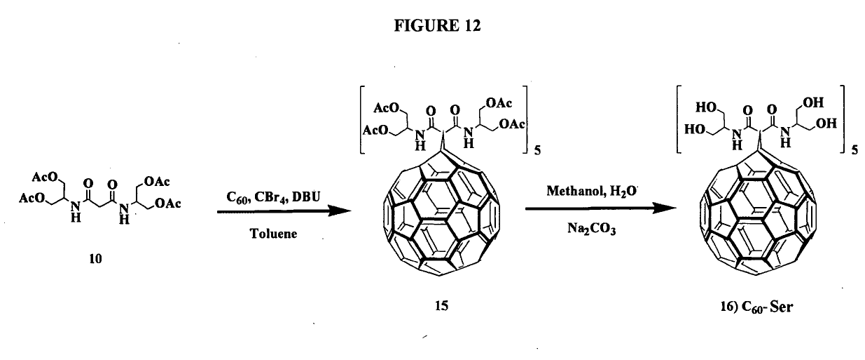

A second water-soluble C₆₀ derivative, 16, without the ability to covalently couple with ZME-018 was prepared for use as a control in the conjugation reaction. This compound was previously reported, with attachment of five addends of 10 to C₆₀ (16 in FIG. 12). The reaction proceeds with addition of 10 to C₆₀ in a 10:1 ratio via in situ Bingel conditions to yield 15. As before, the acetate protecting groups were then removed, leaving water-solubilizing hydroxyl functional groups to obtain 16 (C₆₀-Ser) 16. The antibody coupling reaction was then performed for both C₆₀-SPDP, 14 and C₆₀-Ser, 16.

### Preparation of N,N′-bis[2-(acetyloxy)-1-[(acetyloxy)methyl]ethyl]-malonamide (10)

10 was prepared by slight modifications of a literature procedure. 10.0 g (0.11 moles) of serinol (2-amino-1,3-propanediol) was combined with 7.5 g (0.045 moles) of diethyl malonate and refluxed, using a sand bath, with vigorous stirring at 200-225° C. for 45 min in a 100 mL round bottom flask. The round bottom flask was then removed from the heat to evaporate off the EtOH. The solid, colorless residue was then treated with 40 mL of distilled Ac₂O and pyridine with continuous stirring for 18 hr at room temperature. Finally, 20 mL of chilled MeOH was added carefully to the reaction flask in an ice bath. Solvents were then removed under reduced pressure, and subsequently 75 mL of EtOAc was added. The organic solution was then washed three times with H₂O and saturated NaCl in a 500 mL separatory funnel. The organic layer was dried by contact with MgSO₄, followed by removal of the MgSO₄ by gravity filtration. The EtOAc was removed under reduced pressure to give a yellow solid. The product was further purified by recrystallization from a 2:1 ratio of EtOAc/hexanes to give 12.2 g (0.029 moles) of pure 10 as a white powder; yield 65%. ¹H NMR (400 MHz, CDCl₃) δ (ppm) 2.11 (s, 12H, CH₃), 3.21 (s, 2H, CH₂), 4.21 (m, 8H, CH₂), 4.43 (m, 2H, CH), 7.53 (d, 2H, NH). EI-MS calculated for C₁₃H₂₃O₆N (M⁺) 290.2, found 418.1.

### Preparation of Asymmetric-Protected Amine+Protected Serinol Fullerene (C₆₀) Derivative (11)

100 mg (0.10 mmol) of 7 was dissolved in 100 mL of toluene, followed by sequential addition of 210 mg (0.50 mmol) of 10, 170 mg (0.51 mmol) of CBr₄ and 120 mg (0.78 mmol) of DBU. After stirring overnight, the solvent was removed under reduced pressure and the crude product purified with column chromatography using a 2:3 ratio of acetone/toluene eluant on silica gel to give 70 mg (0.031 mmol) of 11 as a reddish-orange solid; yield 38% based on the trisadduct of 11. MALDI-TOF MS calculated for C₉₀H₄₅O₁₆N₃ (M⁺ bisadduct) 1424, found 1423, calculated for C₁₀₇H₆₉O₂₆N₅ (M⁺ trisadduct) 1840, found 1840, calculated for C₁₂₄H₉₃O₃₆N₇ (M⁺ tetraadduct) 2256, found 2257. For purposes of antibody conjugation, the various isomers of the derivative were not separated.

### Preparation of Asymmetric Amine+Protected Serinol Fullerene (C₆₀) Derivative (12)

70 mg (0.031 mmol) of 11 was dissolved in 100 mL of a 1:1 CH₂Cl₂/TFA solution and stirred for 30 min. The solvent was removed under reduced pressure to give 52 mg (0.030 mmol) of 12 as a reddish-orange solid, yield 97%. MALDI-TOF MS calculated for C₈₅H₃₈O₁₄N₃ (M⁺ bisadduct) 1324, found 1325, calculated for C₁₀₂H₆₂O₂₄N₅ (M⁺ trisadduct) 1740, found 1741, calculated for C₁₁₉H₈₆O₃₄N₇ (M⁺ tetraadduct) 2156, found 2158.

### Preparation of SPDP+ Protected Serinol Fullerene (C₆₀) Derivative (13)

2 mL of TEA was added drop-wise to 100 mg (0.046 mmol) of 12 dissolved in 20 mL of CH₃Cl, followed by addition of 50 mg (0.160 mmol) of SPDP. The solution was then stirred at room temperature overnight. The crude product was purified by column chromatography on silica gel using a 1:1 ratio of toluene/EtOAc eluant. Further purification using HPLC and a 1:1 toluene/EtOAc solvent system was performed to ensure removal of all unreacted SPDP. The final product gave 28 mg (0.012 mmol) of 13 as a brown-red solid; yield 26%. MALDI-TOF MS calculated for C₉₃H₄₄O₁₅N₄S₂ (M⁺ bisadduct) 1520, found 1523, calculated for C₁₁₀H₆₈O₂₅N₆S₂ (M⁺ trisadduct) 1936, found 1940, calculated for C₁₂₇H₉₂O₃₅N₈S₂ (M⁺ tetraadduct) 2352, found 2357.

### Preparation of Water-Soluble SPDP Fullerene (C₆₀) Derivative (14)

Acetate protecting groups were removed from 13 by dissolving 25 mg (0.013 mmol) of 13 in 5 mL degassed methanol, with the subsequent addition of 15 mg (0.137 mmol) Na₂CO₃ and 1.0 mL of degassed DI H₂O under argon. The reddish-orange solution was then stirred for 1.5 h, after which a cation exchange resin (H⁺ form) was added until the solution was pH 7. After an additional 1.0 h of stirring, the solid impurities were removed by gravity filtration and solvent removed under reduced pressure. The crude solid was dissolved in MeOH to perform column chromatography on silica gel with MeOH eluant. The MeOH was then removed under reduced pressure, to give 12 mg (0.006 mmol) of purified 14 as a reddish-orange solid; yield 49%. MALDI-TOF MS calculated for C₉₄H₅₂O₁₇N₆S₂ (M⁺ trisadduct) 1600, found 1620 (M⁺+1 epoxide Os); ¹H NMR (D₂O) was performed to verify that the pyridine moiety was still present on 14 after removal of acetate protecting groups: δ 7.26 (m, 1H), 7.79-7.85 (m 2H), 8.42 (m 1H).

### Preparation of Protected Serinol Fullerene (C₆₀) Derivative (15)

The multi-adduct C₆₀ serinol derivative was prepared by dissolving 50 mg (0.069 mmol) of C₆₀ in 250 mL anhydrous toluene. To this solution, 145 mg (0.35 mmol) of 10, 120 mg (0.36 mmol) of CBr₄ and 85 mg (0.55 mmol) of DBU were added sequentially. The solution was stirred at room temperature overnight to help ensure the maximum degree of functionalization. Toluene was removed under reduced pressure to give a red solid, which was purified by column chromatography using a 4:6 ratio of acetone/toluene eluant on silica gel. After solvent removal, the red solid was dried over P₂O₅ in a drying pistol. This gave 116 mg (0.041 mmol based on the pentaadduct) of purified 15 as a red solid; yield 60%. MALDI-TOF MS calculated for C₁₄₅H₁₂₀N₁₀O₅₀ (M⁺, pentaadduct) 2801, found 2804 (observe M⁺−35, 2769; M⁺−80, 2724; M⁺−122, 2682; M⁺−167, 2637; M⁺−208, 2596; M⁺−248, 2556; M⁺−290, 2514; M⁺−335, 2469 loss off —OAc groups).

### Preparation of Deprotected (Water-Soluble) Serinol Fullerene (C₆₀) Derivative (16)

The serinol functional groups were deprotected by first dissolving 116 mg (0.041 mmol) of 15 in 10 mL of degassed MeOH under Ar. To this solution, 70 mg (0.66 mmol) of Na₂CO₃ and 2 mL degassed DI H₂O were added. The red solution was stirred for 1.5 hr. after which a cation exchange resin (H⁺ form) was added until the solution was pH 7. The solution was then stirred an additional 1 hr and the solvent removed to give 75 mg (0.038 mmol) of 16 as a red solid; yield 93%. MALDI-TOF MS calculated for C₈₇H₄₈O₁₈N₆ (M⁺ trisadduct) 1464, found 1467, calculated for C₉₆H₆₄O₂₄N₈ (M⁺ tetraadduct) 1712, found 1716, calculated for C₁₀₅H₈₀O₃₀N₁₀ (M⁺ pentaadduct) 1960, found 1963.

### Example 3

### Ultra-Short Carbon Nanotube Chemistry

### Malonic acid bis-(3-tert-butoxycarbonylamino-propyl)ester (19)

According to literature procedure, 5.0 g (0.029 moles) of tert-butyl N-(3-hydroxypropyl) carbamate was dissolved in 250 mL anhydrous CH₂Cl₂, followed by addition of 2.0 g (0.014 moles) malonyl chloride. 2.2 g (0.028 moles) of anhydrous pyridine was then slowly added to the reaction vessel. After stirring overnight the reaction was quenched with DI H₂O. The aqueous and organic layers were separated and the organic layer washed three times with DI H₂O. CH₂Cl₂ was then removed under reduced pressure to form a viscous yellow liquid. Further purification of the crude product with column chromatography using a 1:1 ratio of hexane/EtOAc eluant on silica gel gave 3.2 g (0.008 moles) of 19 as a viscous bright yellow liquid; yield 55%. ¹H NMR (400 MHz, CDCl₃) δ (ppm) 1.38 (s, 18H, CH₃), 1.86 (p, 4H, CH₂), 3.19 (q, 4H, CH₂), 3.39 (s, 2H, CH₂), 4.20 (t, 4H, CH₂), 5.30 (s, 2H, NH).

### Reduced and Fluorinated Ultra-Short Single-Walled Carbon Nanotubes (20)

The SWNTs were produced by the high pressure carbon monoxide (HiPco) process.⁷¹ Raw SWNTs were fluorinated in a custom-made flow apparatus using a gaseous mixture of 1% F₂ in He at 50° C. for 2 hr. a condition which gave F-SWNTs with a stoichiometry of CFₓ (x≦0.2).⁷² Under an argon atmosphere, the F-SWNTs were pyrolyzed in a tube furnace at 1000° C., driving off volatile fluorocarbons to yield a chemically-cut ultra-short nanotube (US-tube). Upon cooling, the sample was bath sonicated in concentrated HCl for 1 hr to remove iron catalyst impurities. This process produced bundled US-tubes of average length ˜30 nm, with ˜90% of them shorter than 50 nm⁷³ and residual iron of less than 1.5% by mass. Reduction of the US-tubes was carried out as follows: 30 mg of US-tubes were added to a 250-mL oven-dried round bottom flask, which was then purged with argon. After the addition of 200 mg potassium (or sodium) and 150 mL of anhydrous THF, the reaction mixture was refluxed for 2 hr. followed by 1 hr of sonication. The reduced US-tubes exhibited solubility in THF for 10 days with no visible bundling or precipitation. Excess potassium (or sodium) was removed from the reaction flask in preparation for the Bingel reaction.

Fluorinated US-tubes were prepared using a gaseous mixture of helium-diluted F₂, as described above, at 100° C. The increased temperature was to help ensure maximum fluorination of US-tubes.

### Protected-Amine Functionalized Ultra-Short Carbon Nanotube: US-Tube(Amide) (21)

2 g (0.005 moles) of 19 was added to the reduced US-tube solution from 20 or to 25 mg of fluorinated US-tubes suspended in 150 mL of dry THF in a 500 mL round bottom flask. After sequential addition of 2.5 g (0.008 moles) CBr₄ and 1.5 g (0.010 moles) of DBU the reaction was sonicated for 2 hr and then stirred overnight. The solid was then washed extensively with THF and ether (until a clear wash solution was obtained) on a Pyrex Buchner funnel with a fritted disc to avoid US-tube material affixing to the filter. Finally, the solid was placed in a 35° C. oven and dried overnight to give 15 mg of 21 as a black powder; yield 50%.

One major hurdle, which plagues both SWNTs and US-tubes is the tendency to form bundles, impeding solubility, and thus implementation into biology and medicine. The large π-electron system, which contributes to SWNTs unique electronic properties, has deleterious consequences on solubility. The π-π interaction results in aggregated bundles which have a van der Waals binding energy of ˜0.5 eV per nanometer of tube-tube contact. Debundling and subsequent water suspension of SWNTs has been achieved by wrapping them in polymers, such as polyvinyl pyrrolidone (PVP) and polystyrene sulfonate (PSS), and surfactants, like sodium dodecyl sulfate (SDS). Unfortunately, these methods are not capable of solubilizing US-tubes, as they flocculate from suspension with moderate centrifugation.

The current model of SWNT dispersion postulates that sonication separates SWNTs at tube ends because they contain large length:width aspect ratios, imparting relative flexibility. Once the surfactant or polymer wraps around a tube end, it can propagate along the bundle length, eventually separating into an individual surfactant-coated nanotube. Current belief is that since US-tubes are so small (<50 nm), they act as rigid rods, making this method of debundling futile because there is insufficient torque to peel the tubes off from one another. Therefore an alternative method must be designed to create and isolate individual US-tubes if they ever expect to realize their potential for bioapplications.

Similar to fullerenes the Bingel reaction can be employed to functionalize US-tubes. This allows for further side chain chemistry off the ester or amide Bingel malonate addend, which can be used as a scaffold for various water-solubilizing functional groups, such as amines and hydroxyls. Previously, Bingel addition has been performed on SWNTs using diethyl bromomalonate, making it an ideal candidate to functionalize US-tubes for use in bioapplications. However, for Bingel addition to be effective, both in regards to the degree of functionality and the attainment of single US-tubes, the US-tubes must first be debundled, followed by immediate functionalization, to prevent bundle reformation.

Two strategies to individualize US-tubes, which both allow for subsequent functionalization, were examined, fluorination and alkali reduction. Fluorinated SWNTs have been shown to exfoliate and allow for subsequent nucleophilic substitution (Sₙ1) reactions to occur. A second paradigm, the Birch reduction, uses alkali metals in liquid ammonia to form SWNT salt complexes that are soluble in organic solvents without using sonication, surfactants or functionalization. Attachment of malonate addends to US-tubes was accomplished using in situ Bingel conditions, which enable US-tube functionalization without the complex preparation of bromomalonate, allowing for a variety of malonate addends to be affixed to the US-tubes.

A two-fold strategy was employed to develop Bingel US-tubes. First, the US-tubes are individualized to obtain single US-tubes by either fluorination or reduction, followed by immediate derivatization to prevent rebundling. Here, both methods were compared to determine the extent of exfoliation and the degree of subsequent functionalization.

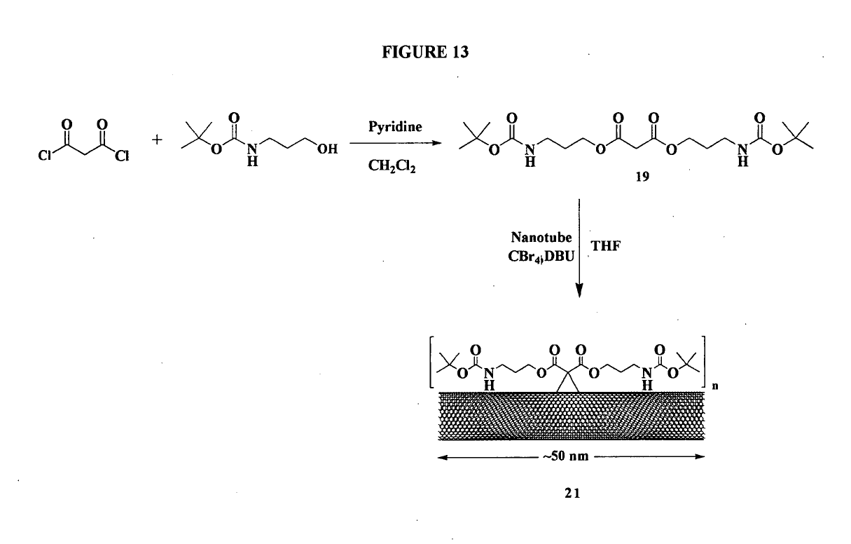

Protected amine functionalized US-tube derivatives, designated as US-tube(Amide), were initially prepared to demonstrate successful implementation of our two-fold US-tube derivatization strategy. First, the malonate addend 19 was synthesized from tert-butyl N-(3-hydroxypropyl)carbamate and malonyl chloride via nucleophilic substitution as shown in FIG. 13. Attachment to individual US-tubes were then accomplished by in situ Bingel addition of 19 to reduced or fluorinated US-tubes to yield the US-tube(Amide) 21. The deprotection of 21 to form the primary amine has yet to be explored. ATR-IR, TGA, XPS and NMR were used to investigate the extent of derivatization on the sidewall of the US-tube(Amide).

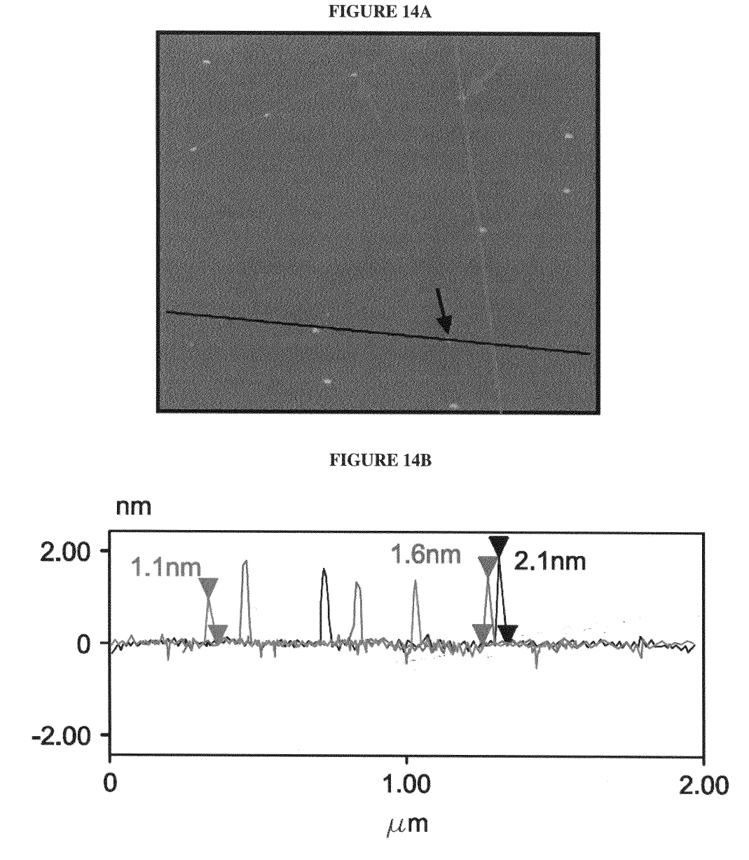

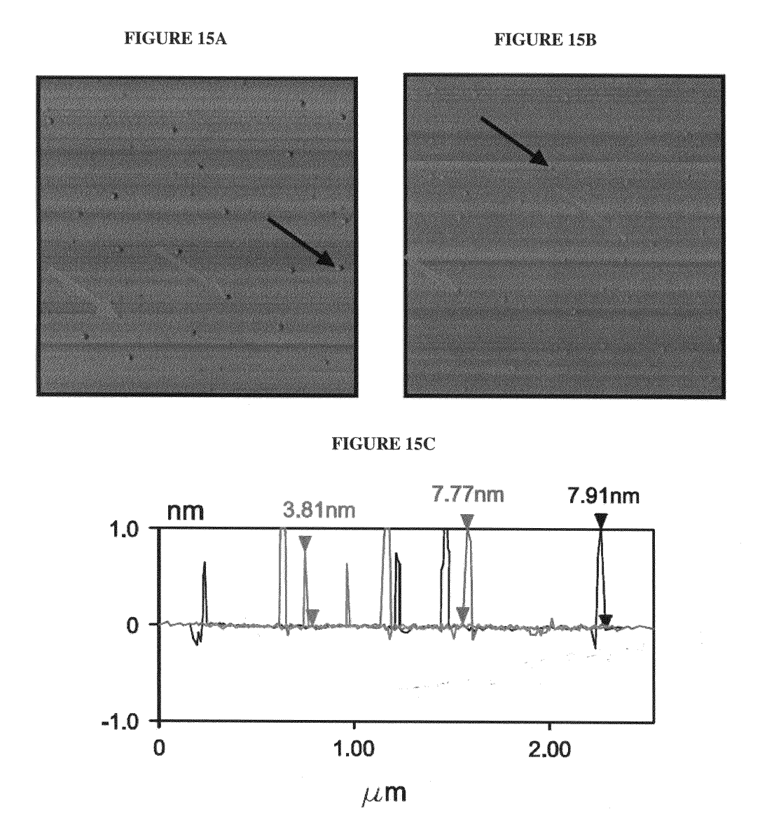

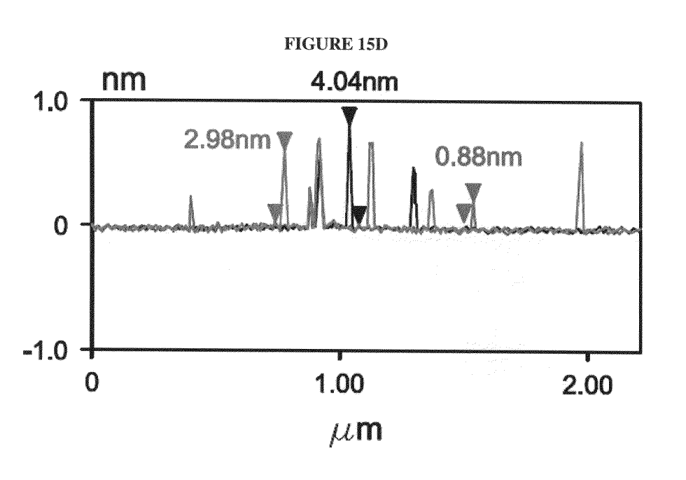

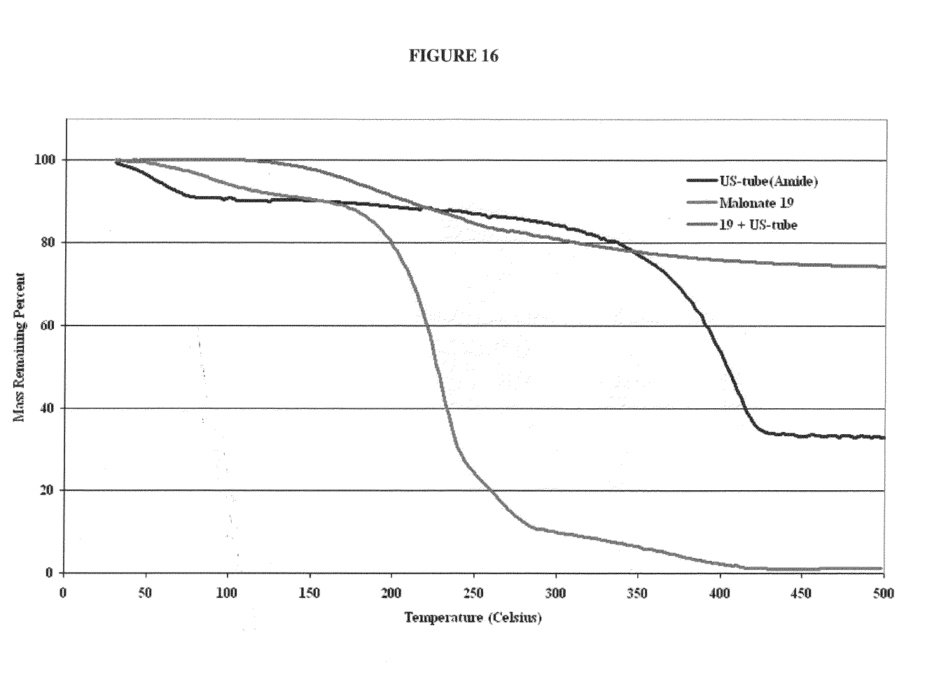

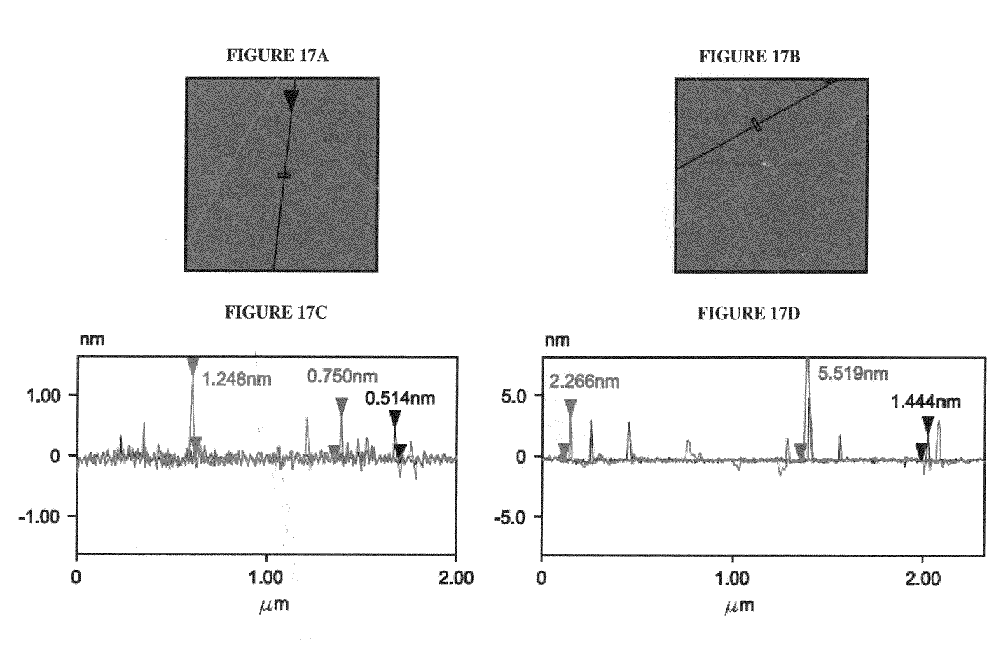

The extent of exfoliation by reduction and fluorination was determined by AFM (FIG. 14 and FIG. 15). Measuring the z-heights of US-tubes and derivatives divulged substantial insight into the relative debundling of each species. A single HiPco tube is on average 1.0 nm diameter, though may vary from 0.5-2.0 nm. Initial AFM analyses of purified US-tubes measured z-resolution heights in excess of 7.0 nm (FIG. 15c)—clearly suggesting a heavily bundled US-tube sample. Subsequent AFM analysis on fluorinated-US-tubes showed z-resolution heights ranging from single US-tubes of 1.4 nm to significantly bundled US-tubes of over 5.5 nm (FIG. 16d). In comparison, reduced US-tubes showed z-resolution heights ranging from 0.5-1.5 nm, corresponding to single US-tubes (FIG. 16c). Clearly, reduction produced the most efficient debundling of US-tubes. The US-tube(Amide) derivatives mirrored debundling data with reduced US-tubes manifesting z-heights ranging from 1.1-2.1 nm (FIG. 14), while US-tube(Amide) derivatives from fluorinated US-tubes revealed z-height ranges from 0.9-4.0 nm (FIG. 15d).

It would be expected that reduced US-tubes would functionalize to a greater extent due to their greater exposed surface area. However, elemental XPS data indicates the contrary. XPS analysis shows the presence of Bingel functionalization as measured by increased nitrogen-content resulting from Bingel addition of 19 to US-tubes (Table 1). For comparison, the Bingel reaction was performed on purified US-tubes, demonstrated moderate functionalization with an increase in nitrogen atomic percent of 1.5%. Both the fluorinated US-tube(Amide) (increase 4.5%) and reduced US-tube(Amide) (increase 3.1%) derivatives comprised a greater degree of functionalization relative to pure US-tubes, due to the inherent debundling of the reduced and fluorinated US-tubes. Subsequent reactions on US-tube(Amide) found that the nitrogen percent plateaus at about 5%. Assuming that 1 nm of US-tube consists of 120 carbons, it is calculated that approximately 4-5 Bingel malonate groups are attached per nm of US-tube.

TABLE 1
XPS analysis of US-tubes and derivatives
(±0.5%, numbers in atomic %)

| Sample | C % | F % | N % | K % | O % |
|---|---|---|---|---|---|
| US-tubes | 90.4 | 0.9 | 0.5 | 0.0 | 8.0 |
| Reduced US-tubes | 75.3 | 0.6 | 0.4 | 12.5 | 10.5 |
| Fluorinated US-tubes | 71.2 | 18.7 | 0.5 | 0.0 | 9.6 |
| US-tube (Amide) | 91.8 | 0.3 | 2.0 | 0.0 | 5.9 |
| Fluorinated US- | 80.7 | 5.3 | 5.0 | 0.0 | 9.3 |
| tube (Amide) | | | | | |
| Reduced US- | 86.4 | 0.3 | 3.5 | 1.2 | 8.1 |
| tube (Amide) | | | | | |

The greater functionalization of fluorinated US-tubes over reduced US-tubes can be attributed to the electron-withdrawing character of fluorine. The Bingel malonate addend behaves as a nucleophile, reacting favorably with electron deficient carbons. The reduced US-tubes are coated with ˜10 e⁻/nm, which causes an electrostatic repulsion to exfoliate US-tubes, but impedes the nucleophilic Bingel addition of malonate addends to the US-tubes. Conversely, the fluorinated US-tube incorporates no additional negative charge, while possessing an abundance of electron-withdrawing fluorine atoms. The fluorine attached to the US-tube acts as an electron sink, causing an increase in electropositive character at the reaction site, creating an environment conducive for Sₙ1-reactions. A second hindrance exhibited by reduced US-tube is the tendency for negatively charged species to undergo hydrogenation, promoting C—H bond formation, which has previously been observed with reduced SWNTs. Hydrogen ions are produced during the in situ bromination of 19, which can compete for reaction sites on the reduced US-tube, in effect diminishing the reaction sites available for attachment of 19, accounting for the lesser degree of functionalization exhibited by the US-tube(Amide) from the reduced US-tubes.

The reduced US-tube(Amide) was characterized by ATR-IR, TGA and NMR. The ATR-IR spectrum confirms the presence of carbonyl functionality due to the strong C═O stretch at 1736 cm⁻¹. This corresponds to the carbonyl ester groups from attached malonate 19. Degradation of the US-tube(Amide) is evident in the TGA plot as the temperature is ramped to 350° C. (FIG. 16). This is characteristic of side-chain cleavage of the malonate from the US-tube(Amide). TGA was performed on a US-tube/malonate mixture (not covalently attached) for comparison, in which the malonate volatilized at 200° C., confirming that the loss of mass in the US-tube(Amide) is indeed from covalently-attached malonates of the US-tubes. After cleavage, approximately 40% of the US-tube mass remains, indicating that approximately 60% of US-tube(Amide) mass is contributed from the Bingel malonate addends. This is consistent with XPS data that calculated the attachment of approximately 4-5 Bingel malonates per nm of the US-tubes.

The basic ¹H-¹³C cross-polarization/magic-angle spin (CP-MAS) spectra was acquired with 7 kHz MAS, a 1-ms contact time, 29.3-ms free induction decay (FID), and 5-s relaxation delay. The FID after 48,400 scans was processed with 50 Hz (1 ppm) of line broadening. The dipolar-dephasing spectrum differed only in that after CP; two 25-μs dephasing periods with a 180° ¹³C refocusing pulse in the middle were used before FID acquisition in order to eliminate the methylene signals. The FID obtained after 67,600 scans was processed with 50 Hz of line broadening. Chemical shifts are reported relative to the carbonyl carbon of glycine defined as 176.46 ppm.

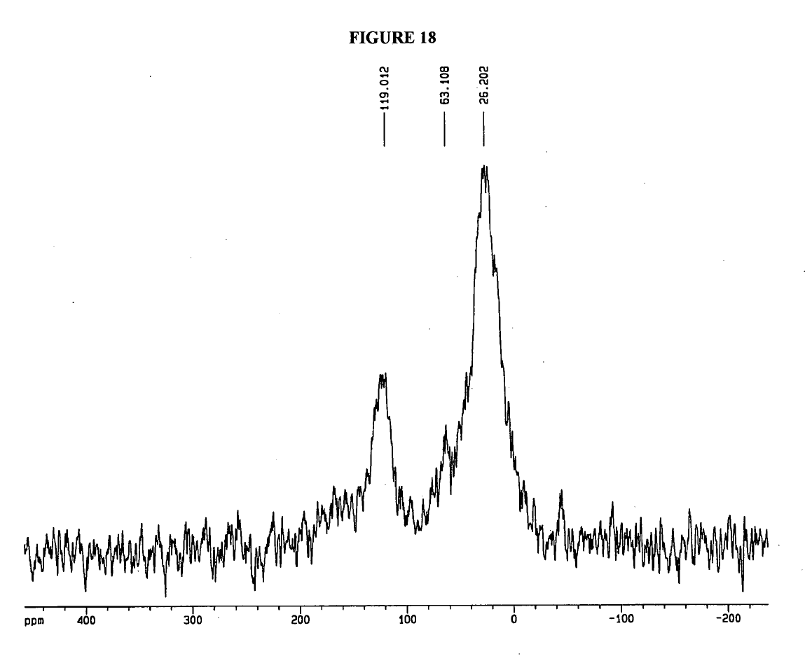

The basic ¹H-¹³C CP-MAS spectra (FIG. 18) indicate sp³ and sp² functionality. The upfield portion of the aliphatic signal results from overlapping signals from the tert-butyl methyl carbons and two of the three different types of methylene carbons. A peak maximum of 26 ppm is upfield of what would be expected for such carbons and indicates that the US-tube is exerting a shielding effect on the addend. The downfield tail of the aliphatic signal is consistent with overlapping signals from the different quaternary carbons of the cyclopropane ring, the methylene carbon adjacent to oxygen, and the tert-butyl quaternary carbon (also adjacent to oxygen). The carbons of the cyclopropane ring can be expected to give relatively weak signals in light of their distance from the nearest protons, while the tert-butyl quaternary carbon can be expected to give a relatively weak signal resulting from weak ¹H-¹³C dipole-dipole interactions with the highly mobile methyl protons. The prominent sp² signal at about δ120 clearly results from unfunctionalized sp² carbons of the US-tube, while its downfield tail is consistent with overlapping signals from the carbamate and ester carbonyl carbons. The signal at about δ120 can reasonably arise from cross polarization from methylene protons of the addend lying along the US-tube, a particularly clear example of the through-space nature of cross polarization.

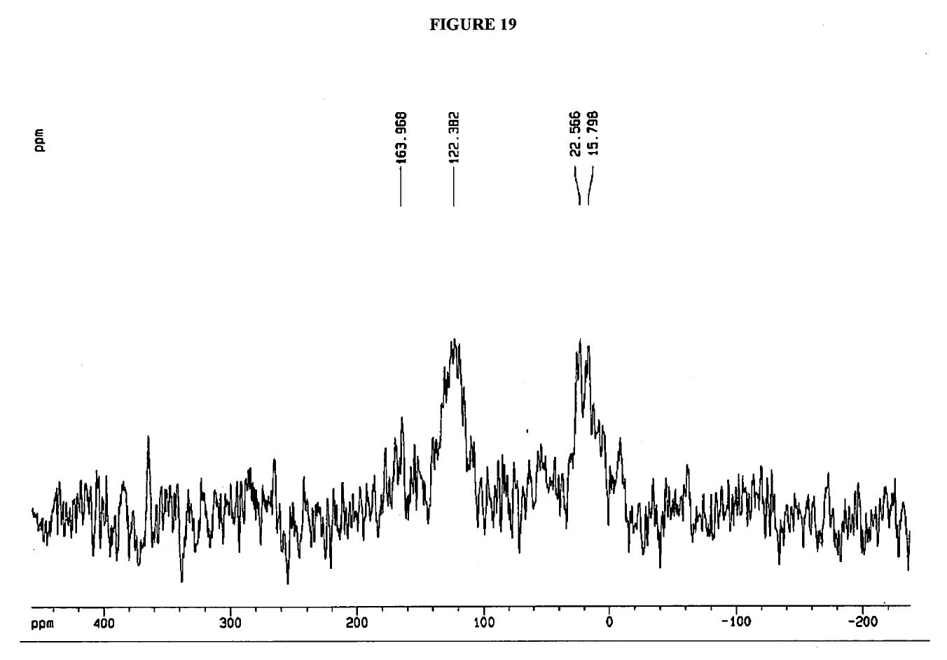

The CP-MAS spectrum with a pair of 25-μs dephasing periods (FIG. 19) displays only attenuated signals from methyl and quaternary carbons. The tert-butyl methyl signals are clearly weak after only 50 μs of dephasing; this may reflect only partial cross polarization with just a 1-ms contact time before the dephasing process. Lengthening the contact time to 3 ms did not result in a detectable aliphatic signal after 17,400 scans, which suggests that T_{1ρ}(H) is no more than a few milliseconds. Regardless, peak maxima at about 15-20 ppm are clearly upfield of what would be expected for tert-butyl methyl carbons, as these signals are at δ29 in the precursor malonate or correspondingly functionalized C₆₀. The other types of quaternary aliphatic carbon would definitely give signals further downfield. Therefore, the US-tube is obviously exerting a shielding effect on the addend. It can be speculated that the malonate functional group is tightly wrapped around the nanotube, which contains a small residual negative charge from the reduction reaction. This could account for the shielding of the methyl signals. This NMR data strongly suggests that the formation of covalently functionalized US-tube(Amide) was accomplished.

### Example 3

### Biocompatible US tubes

### Protected-Serinol Functionalized Ultra-Short Carbon Nanotube (22)

Reduced US-tubes were functionalized with 10 using the same methodology as for compound 21.2 g (0.005 moles) of 10 was added to the reduced US-tube solution from 20 in anhydrous THF. While sonicating, 2.5 g (0.008 moles) of CBr₄ and 1.5 g (0.010 moles) of DBU were added sequentially to the reaction flask, sonicated an additional 1 hr and then stirred overnight. The solid was washed with THF and ether similar to 21 and dried overnight in a 35° C. oven. The total amount of 22 recovered was 15 mg; yield 50%.

### Deprotected (Water-Soluble) Serinol Functionalized Ultra-Short Carbon Nanotube: US-Tube(Ser) (23)

Acetate protecting groups were removed by sonicating 25 mg of 22 in 50 mL of degassed MeOH for 1 hr. followed by addition of 500 mg Na₂CO₃ and 5 mL of degassed DI H₂O. The solution was then sonicated for 1.5 hr after which cation exchange resin (H⁺ form) was added until the solution was pH 7. The solution was then sonicated for an additional 1 hr. The Na₂CO₃ was removed by washing the US-tube(Ser) three times with DI H₂O, with subsequent centrifugation in a 3200-rpm centrifuge and removing the supernatant, which contained the Na₂CO₃. The total amount of 23 recovered was 6 mg; yield 24%.

### Diethyl Malonate Functionalized US-Tube: US-Tube(Ester) (24)

Reduced US-tubes were functionalized with diethyl malonate using the same methodology as compound 21 with slight modifications. 50 mg (0.21 mmol) of diethyl bromomalonate was added to 20 mg of reduced US-tubes in a 1:1 ratio of anhydrous toluene/THF solvent system under argon. While stirring, 50 mg (2.0 mmol) of NaH was added to the reaction flask and allowed to stir overnight. The solid was then collected and washed with EtOH and H₂O on a PTFE filter to remove excess NaH. After washing the solid was placed in a 35° C. oven to dry overnight. The total amount of US-tube(Ester) recovered was 12 mg; yield 60%.

### Carboxylic Acid Functionalized Ultra-Short Nanotube: US-Tube(COOH) (25)

Hydrolysis of 24 was accomplished by suspending 20 mg of 24 in 5 mL MeOH, followed by the addition of 5 mL 1 M NaOH. The solution was then stirred at room temperature (avoid decarboxylation) for 24 hr; yield 100%.

### PEG Functionalized Ultra-Short Nanotubes: US-Tube(Peg) (26)

1.0 mL (0.011 moles) of oxalyl chloride was added directly to the solution in 25 and sonicated for 24 hrs under Ar. 1.0 mL of PEG, which had been dried over P₂O₅, was then added to the reaction flask and condensed at 120° C. for 5 days. The solid was collected and washed with EtOH on a PTFE filter to give 9 mg of US-tube(PEG); yield 20%.

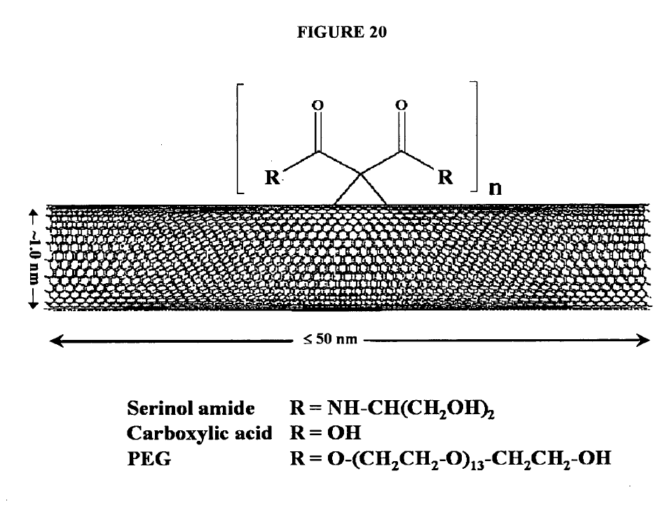

Biologically compatible, empty US-tube materials (FIG. 20) were developed. The US-tube nanocapsules have been individualized using the same Na⁰/THF reduction procedure and Bingel derivatization used in synthesizing the individual US-tube(Amide).

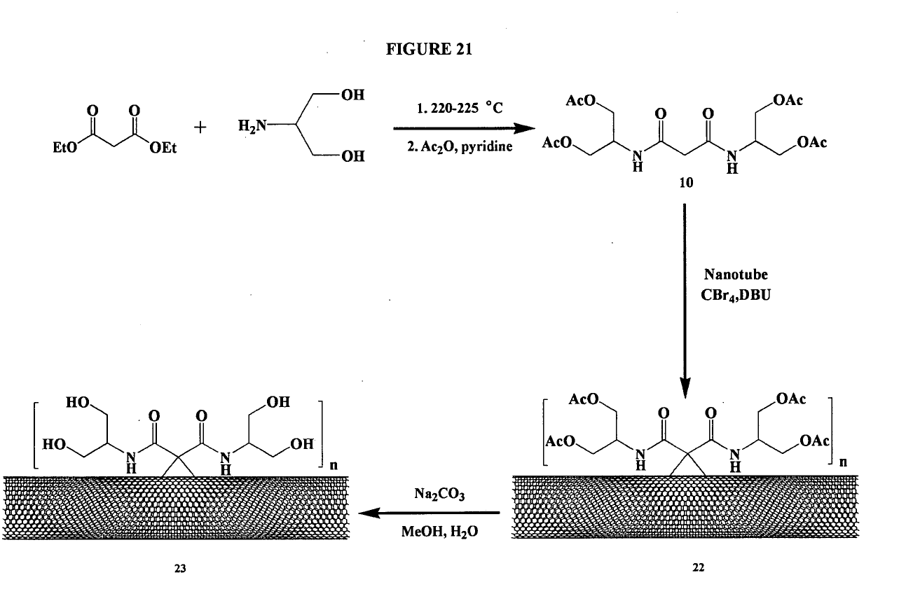

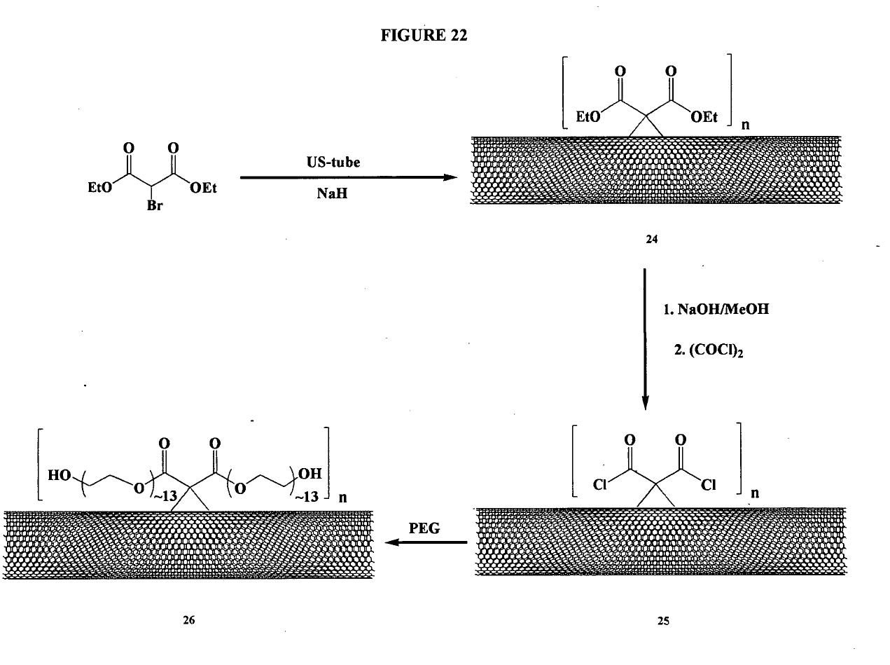

The US-tubes were prepared, purified and reduced as discussed in experimental section, then functionalized (R groups in FIG. 20) with carboxylic acid, serinolamide (FIG. 21) and PEG (FIG. 22) moieties using in situ Bingel reaction conditions. The Bingel conditions produce protons, which undoubtedly protonate, and thus competes for reaction sites on the reduced US-tubes, in a similar manner to when reduced SWNTs are quenched with MeOH or water.

Biocompatible serinol functionalized US-tubes, designated as US-tube(Ser) were prepared by in situ Bingel addition of 10 to form 22. Subsequent cleavage of the acetate protecting groups gave the US-tube(Ser) US-tube derivative 23. PEG US-tubes, designated as US-tube(PEG) were prepared using a modified Bingel procedure. Diethyl malonates were attached to the US-tubes from the bromomalonate and NaH to form 24. The diethyl esters were then hydrolyzed to produce carboxylic acid functionalized US-tubes, designated as US-tube(COOH) which were converted to the acid chloride 25 using oxalyl chloride. Finally, PEG was attached to the US-tube through a nucleophilic substitution of the acid chloride to yield 26.

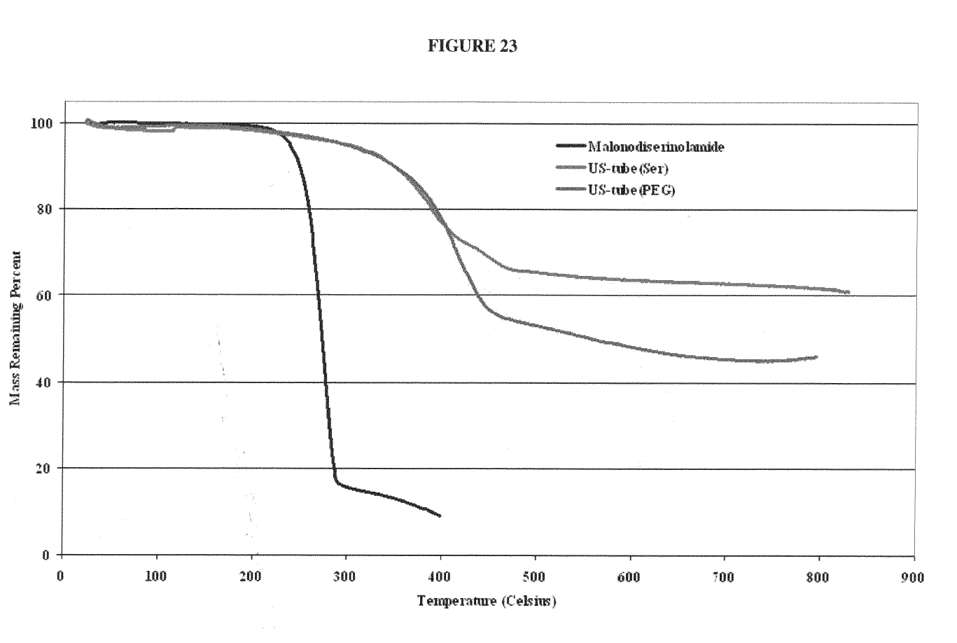

The degree of functionalization and exfoliation of the US-tube derivatives were determined using XPS, TGA and AFM. XPS was used to confirm that functionalization occurred. The atomic percent nitrogen in unfunctionalized US-tubes is ≦0.5%, but after the Bingel reaction with protected malonodiserinolamide, the atomic percent of nitrogen increased to ˜6.0%. This can be attributed to the amide functionalities from the nitrogen on malonodiserinolamide. Assuming that the average US-tube contains 120 carbons/nm, approximately 5% of the US-tube was functionalized. TGA was also performed on the US-tube(Ser) sample and found that the mass gradually decreased from 350-500° C. (FIG. 23). The free serinol malonate showed a sharp decrease in mass at 250° C., confirming covalent bond attachment of the malonodiserinolamide adduct. For comparison, a TGA of US-tube(PEG) was obtained (FIG. 23), showing a gradual mass loss of approximately 55%, which agrees with the amount of functionalization observed by the US-tube(Ser). This also implies that the US-tube(COOH) derivatized to a similar extent. This degree of functionalization compares favorably with previous work that determined SWNT-PEG graft polymers functionalized 1% of carbons and SWNT-PABS 4% of carbons.

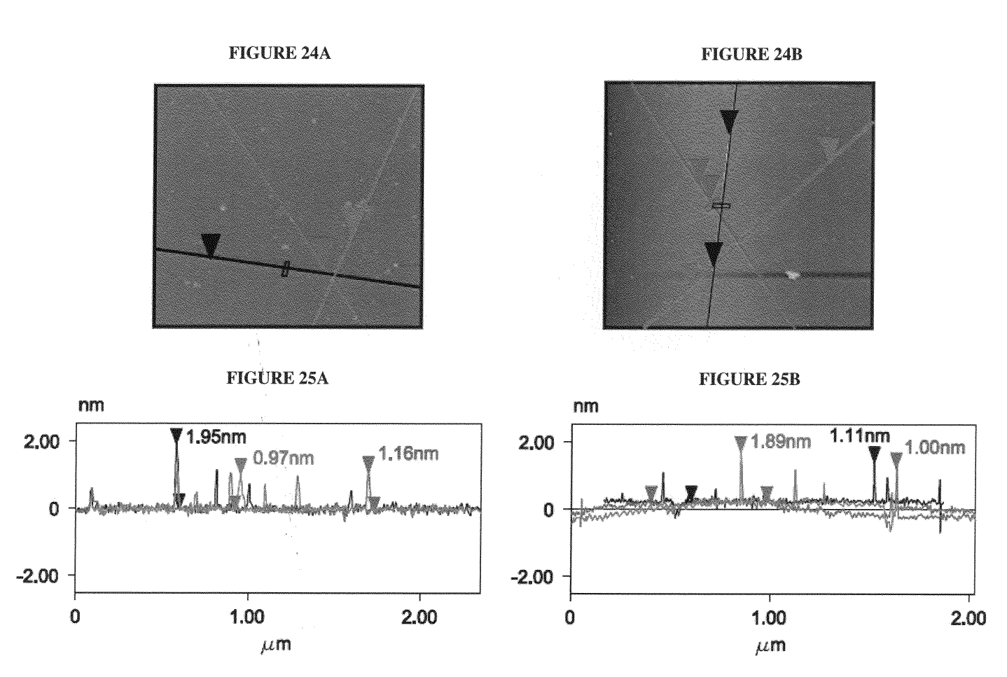

Tapping-mode AFM was used to show that exfoliation of US-tube(Ser) and US-tube(PEG) occurred. AFM images (FIG. 24) and z-scan analyses (FIG. 25) illustrated that indeed the US-tube(Ser) and US-tube(PEG) materials had been individualized. The z-height analyses of the two US-tube samples displayed ranges from 0.97-1.79 nm for US-tube(Ser) and 1.00-1.89 for US-tube(PEG), which coincide with diameters of individual HiPco US-tubes (0.5-2.0 nm)¹¹⁸ tubes with an expected slight increase in height as a result of the functionalization. In addition, it can be seen that over 90% of the functionalized US-tubes have heights that correspond to individualized tubes, with the remaining fraction corresponding to small bundles.

The water solubility and partition coefficients (K_{ow}) of functionalized US-tubes were determined using UV-vis-NIR spectroscopy at a physiological pH of 7.4 (Table 2). Samples were dissolved in water at several concentrations up to 2.0 mg/mL. The absorbance of each sample was then determined as the spectra were recorded sequentially. The solubility was taken as the point at which the absorbance ceased to increase in intensity linearly with concentration. This method produced solubilities of 1.00 mg/mL for the US-tube(PEG), 0.25 mg/mL for US-tube(Ser) and 0.05 mg/mL for US-tube(COOH). Each of the 2.0 mg/mL samples were centrifuged at 3200 rpm for 30 min, whereby both the US-tube(Ser) and US-tube(COOH) samples spun down. In contrast, the US-tube(PEG) sample remained in solution at an impressive 0.50 mg/mL (as measured by UV-vis). In a separate experiment, free PEG was added to pristine, individualized US-tubes in water and the mixture was sonicated for one hr. After sonication, this PEG/US-tube mixture showed no solubility (colorless solution) and all the US-tube material spun down when centrifuged. This result established that the PEG groups in the US-tube(PEG) sample are indeed covalently attached to (and not just physically wrapped around) the US-tube.

The n-octanol/water partition coefficient (K_{ow}), which is useful in the determining biological structure-activity relationships, was obtained from K_{ow}=cₒc_{w}⁻¹, where cₒ and c_{w} are the equilibrium concentrations of the analyte in n-octanol and water, respectively, at 25° C.^{66,130} A 0.25 mg/mL solution of each of the three US-tube samples was shaken with an equal volume of n-octanol and water. The UV-vis absorbance of the aqueous layer and organic layer were then measured independently for each sample. In the case of US-tube(PEG), K_{ow}=1.21, for US-tube(COOH), K_{ow}=0.83 and for the US-tube(Ser), K_{ow}=0.26 at pH=7.4. In comparison, K_{ow}=0 for a malonodiserinolamide derivative of C₆₀. A K_{ow} value of 0, which indicates negligible lipophilicity, is typical of drugs which are restricted to extracellular space and rapidly clear from the body. This data suggests that the most lipophilic US-tube derivative, US-tube(PEG), would likely internalize into cells. Even the US-tube(Ser) agent, with the lowest K_{ow} value (0.26) in Table 2, would also likely internalize, since a similar K_{ow} value for a polyarginine-containing Gd(DOTA) MRI CA resulted in internalization.

TABLE 2
Water solubility and n-octanol/water partition coefficient (K_{ow}) for
three derivatized US-tubes species at pH = 7.4

| | Solubility | |
|---|---|---|
| | (mg/mL) | K_{ow} |
| US-tube (PEG) | 1.00 | 1.21 |
| US-tube (Ser) | 0.25 | 0.26 |
| US-tube (COOH) | 0.05 | 0.83 |

### Example 3

### Antibody Chemistry

### Preparation of C60-SPDP and C60-serinol ZME-018 Immunoconjugates (17,18)

2.0 mg of ZME-018 mAb was added to 3.4 mL of phosphate/saline buffer. TEA was then added until pH 8.0, followed by the addition of 1 mM H₄EDTA. Free thiol functionalities were then attached to the antibody with addition of 7.8 μL 2-iminothiolane to the above solution with constant stirring under nitrogen at 4° C. for 90 min. Non-reacted 2-iminothiolane was removed with a G-25 sephadex size-exclusion column using an eluant consisting of 5 nM bis/tris, 50 mM NaCl and 1 mM H₄EDTA at pH=5.8. Fractions containing the thiol-derivatized antibody were determined using a Bio-Rad protein assay. The antibody fractions were then combined and pH brought to 7.0 with TEA. The antibody solution was halved to allow for immunoconjugation with both the C₆₀-SPDP and C₆₀-Ser samples. 123.7 μL C₆₀-SPDP and 130.8 μL C₆₀-Ser were each added to one of the resulting antibody solutions (10:1 C₆₀:antibody) and stirred overnight at 4° C. A white solid of unreacted antibody precipitated out of the solution during the night. This solid was removed by centrifugation. The immunoconjugates were then purified with a G-25 sephadex size-exclusion column using a buffer of 10 mM Na₃PO₄ and 140 mM NaCl at pH=7.2 to remove any non-conjugated C₆₀ material from the sample. A Bio-Rad protein assay was utilized to determine which fractions contained the immunoconjugate. Aliquots of the purified immunoconjugates were taken and dialyzed overnight in 6 M Urea to ascertain whether any covalent linkages formed between the C₆₀-SPDP and antibody. Several analytical techniques were implemented in the characterization of the immunoconjugates, including triplet-triplet absorption, UV-vis, transmission electron microscopy (TEM), and Bio-Rad protein assays. These are discussed in more detail below.

Enzyme-linked immunosorbent assay (ELISA) was performed to determine if the C₆₀-immunoconjugates retain specificity to the A375m melanoma cells. ELISA plates were prepared by versene-stripping 50,000 gp240-antigen-positive A375m melanoma cells from tissue culture flasks, which were washed 2 times with DPBS. The cells are then rehydrated in DPBS in the individual wells of a Falcon 3912 96-well μl-plates, leaving 2 empty wells for blanks. The plates were dried overnight at 37° C. and stored at 4° C. until used. The ELISA was initiated by adding 200 μl of blocking buffer to each well with incubation for 1 hr at room temperature. The blocking buffer was removed by decanting, followed by immediate addition of 100 μl/well of various antibody standards and unknowns. The plate was incubated for 3 hr at room temperature and solution removed. Each well was washed three times with a washing buffer for preparation of IgG component detection. Concurrently, anti-mouse IgG-HRP was diluted 1:1000 in a dilution buffer, making 11 ml/plate. A 100 μl/well aliquot was added to the cells and incubated for 15 min at room temperature. The wells were then washed three times with a washing buffer. Simultaneously, 11 μl of H₂O₂ was added to 11 ml of ABTS, which was added 100 μl/well to the cells and incubated for 10 min at room temperature. The plate was then read at 405 nm to plot the ELISA binding curve in order to calculate the IC(50) values.

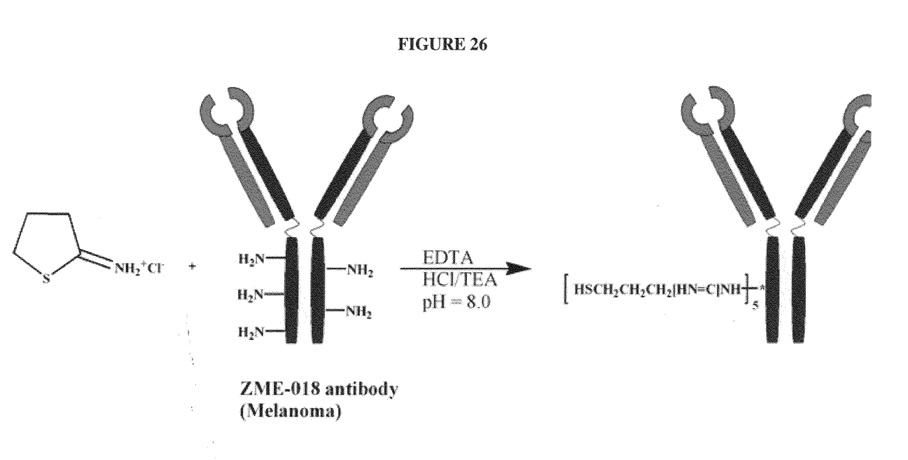

Preparation of the ZME-018 mAb for coupling to 9 was achieved by attachment of a free-thiol arm to the ZME-018 mAb using 2-iminothiolane (FIG. 26). Nucleophilic attack on the electropositive carbon atom adjacent to the iminium ion allowed for the primary amines from the antibody to sever the C—S bond, thus liberating the alkyl thiol, which is necessary for covalent coupling to C₆₀-SPDP derivative. On average, five thiol functionalities are attached to the antibody using this method. Non-reacted 2-iminothiolane is removed from the thiol-containing antibody by size exclusion chromatography.

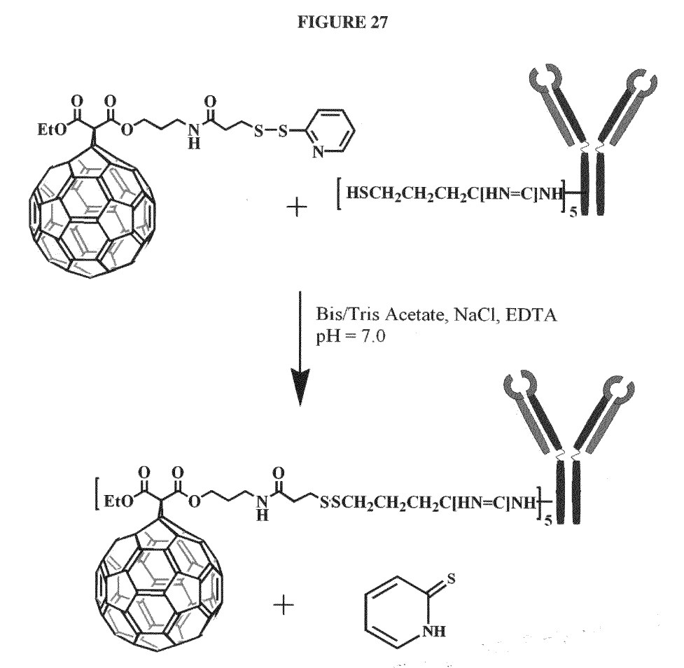

The coupling of 9 with the ZME-018 mAb occurred by reacting 9 with the ZME-018 solution at pH 7.0. The conjugation reaction was stirred overnight to allow for the new disulfide linkage between C₆₀-SPDP and the ZME-018 mAb to form, with concurrent release of 1H-pyridine-2-thione (FIG. 27). Unfortunately, after stirring overnight, a precipitate was observed consisting of unreacted 9 and denatured ZME-018 mAb, with no indication of conjugate formation. This suggested that 9 was not sufficiently water soluble for coupling to ZME-018.

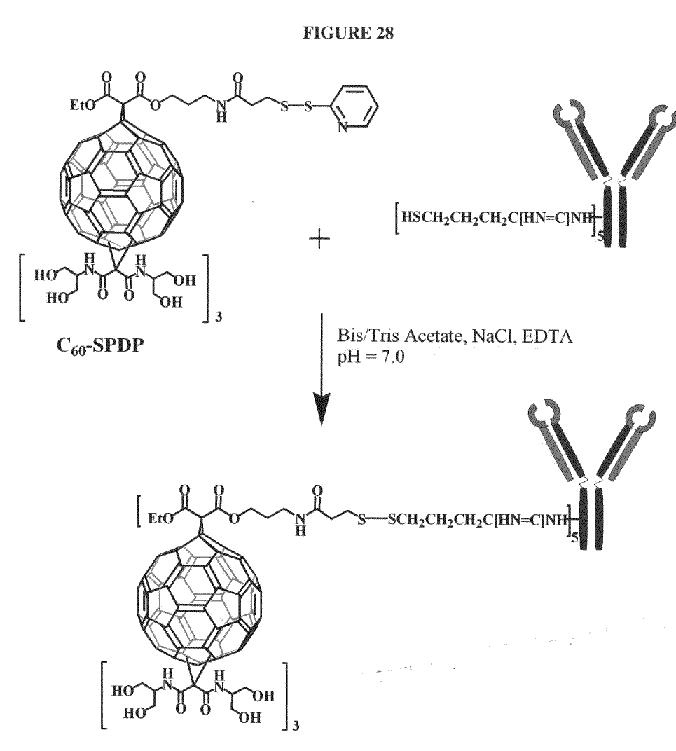

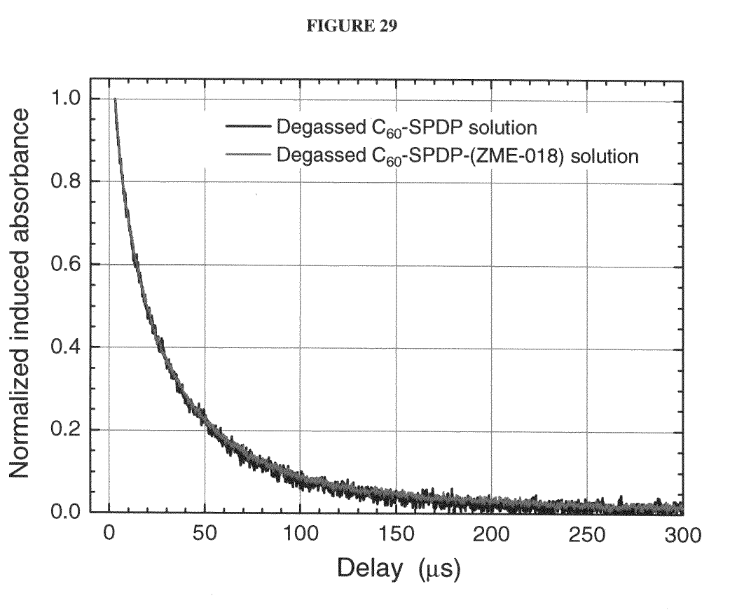

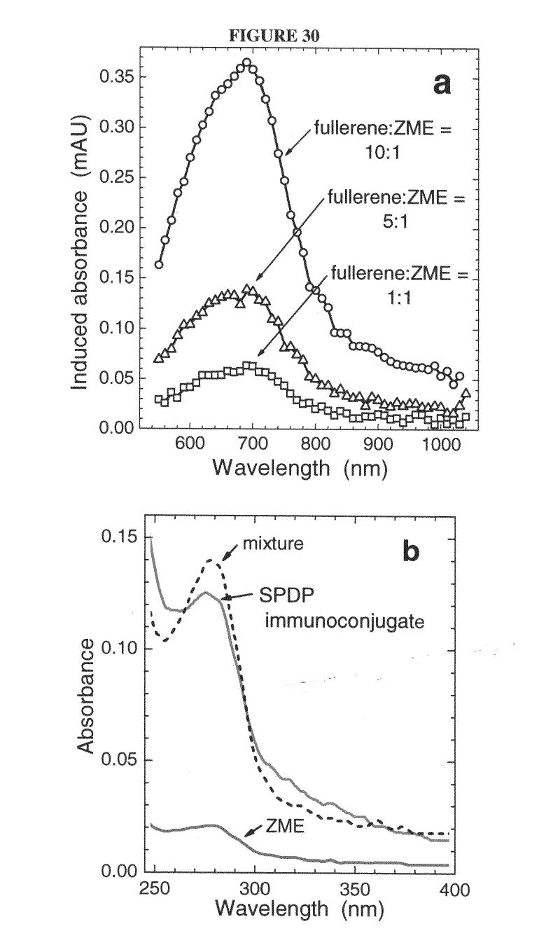

The two C₆₀ derivatives, C₆₀-SPDP 14 and C₆₀-Ser 16 were successfully conjugated to the ZME-018 mAb. Coupling of C₆₀-SPDP to the antibody (for ratios of 1:1, 5:1 and 10:1) was accomplished by reacting the thiol derivatized ZME-018 mAb with the SPDP sidearm of C₆₀-SPDP (FIG. 28). The coupling was performed in a salt solution to minimize fullerene aggregation.⁸⁹ Products were purified by size-exclusion chromatography and then examined by transient absorption spectroscopy (FIG. 29). As shown in FIG. 30a, the C₆₀ core's 690 nm triplet-triplet spectral signature was clearly present with intensities reflecting the reactant ratio. This technique was utilized as proof that C₆₀ material did in fact interact with the ZME-018 mAb, but not to quantify the amount of C₆₀ enclosed within the immunoconjugate. Unfortunately, it was unclear whether covalent bonds had formed between C₆₀-SPDP and the ZME-018 mAb. Therefore, the related water-soluble C₆₀-Ser derivative (16), was substituted for C₆₀-SPDP in the reaction schemes with ZME-018 mAb (10:1 C₆₀-Ser:ZME-018). To our surprise, results for the C₆₀-Ser derivative mirrored those of C₆₀-SPDP. This implies that C₆₀-antibody conjugate formation may not require covalent bond formation.

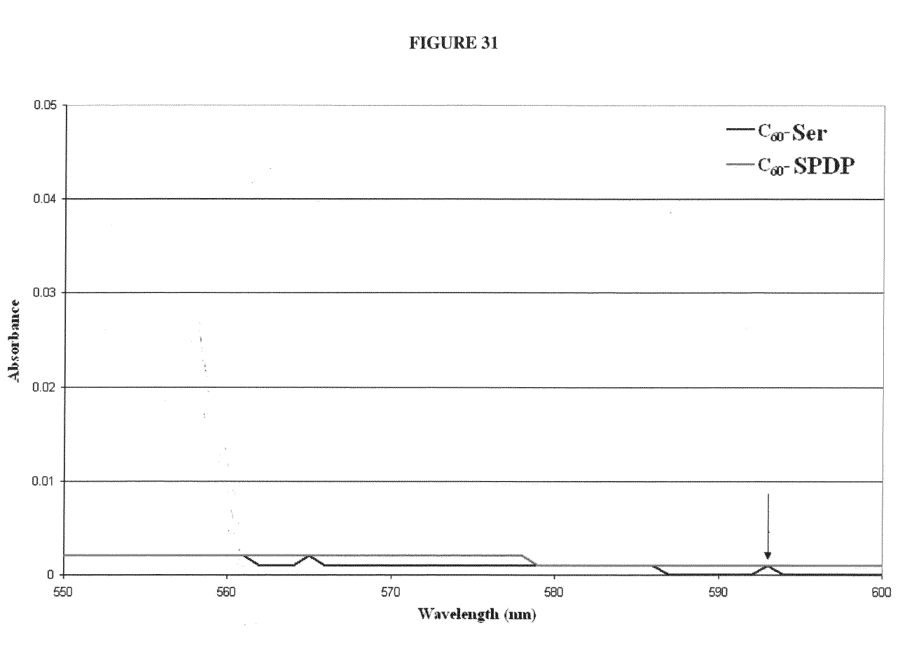

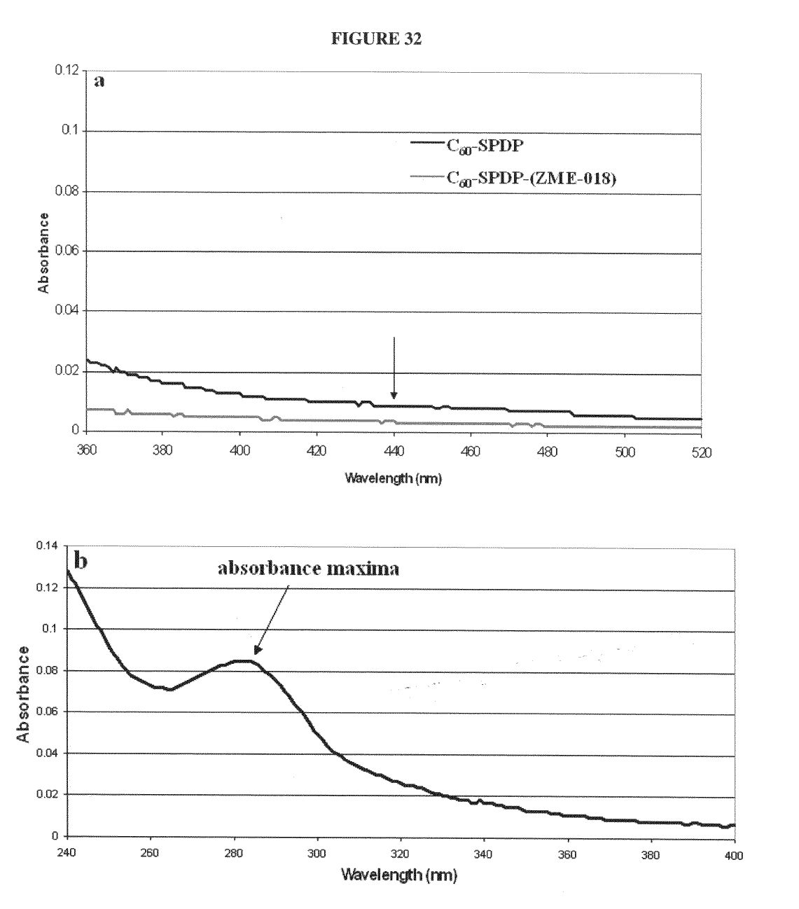

Quantitative characterization began with Bio-Rad protein assays, which use UV-vis spectroscopy at 595 nm (no C₆₀ interference as shown in FIG. 31) that showed the concentration of ZME-018 in the chromatographically purified samples as 0.40 μM for C₆₀-SPDP-(ZME-018) and 0.36 μM for C₆₀-Ser-(ZME-018). To find the corresponding fullerene concentrations in these conjugates, we used UV-vis spectroscopy. At 440 nm, the molar absorptivity of C₆₀-Ser far exceeds that of ZME-018. The conjugate's measured 440 nm absorbance directly showed a C₆₀-Ser concentration of 15 μM, implying that the ratio of C₆₀-Ser:ZME-018 was 38:1. Spectral analysis of the C₆₀-SPDP-(ZME-018) conjugation was more complex because absorption bands of C₆₀-SPDP at 440 nm (FIG. 32) are not intense enough for determining concentrations of <20 μM and at lower wavelengths (<350 nm) there is an overlap from absorption bands from the ZME-018 mAb. To account for this, we first prepared a reference solution containing only 0.40 μM ZME-018. As shown in FIG. 30b, this solution has significant absorption at 282 nm (this is an absorption maxima of the C₆₀-SPDP derivative as shown in FIG. 32). C₆₀-SPDP was then added until the absorbance of the mixture near 282 nm matched that of the C₆₀-SPDP-(ZME-18) immunoconjugate known to contain a 0.40 μM concentration of antibody. The upper traces in FIG. 30b show spectra of this mixture and the conjugate. From the amount of C₆₀-SPDP used to prepare the matching mixture, we deduced a C₆₀-SPDP concentration of 6 μM in the conjugate, corresponding to a C₆₀-SPDP:ZME-018 molar ratio of 15:1. Urea dialysis was performed on both the C₆₀-SPDP-(ZME-018) and C₆₀-Ser-(ZME-018) immunoconjugates in an attempt to determine if any C₆₀-SPDP was covalently attached to the ZME-018 mAb. Urea denatures proteins, which would theoretically cause release of any non-covalently linked C₆₀ material from the ZME-018 mAb, while retaining covalently attached C₆₀. However, after dialysis, both the C₆₀-SPDP (50% loss) and C₆₀-Ser (60% loss) immunoconjugates displayed a reduction in C₆₀ concentration. Even though C₆₀-Ser displayed a slightly greater reduction compared to C₆₀-SPDP, it was inconclusive whether C₆₀-SPDP-(ZME-018) contained any covalent attachment.

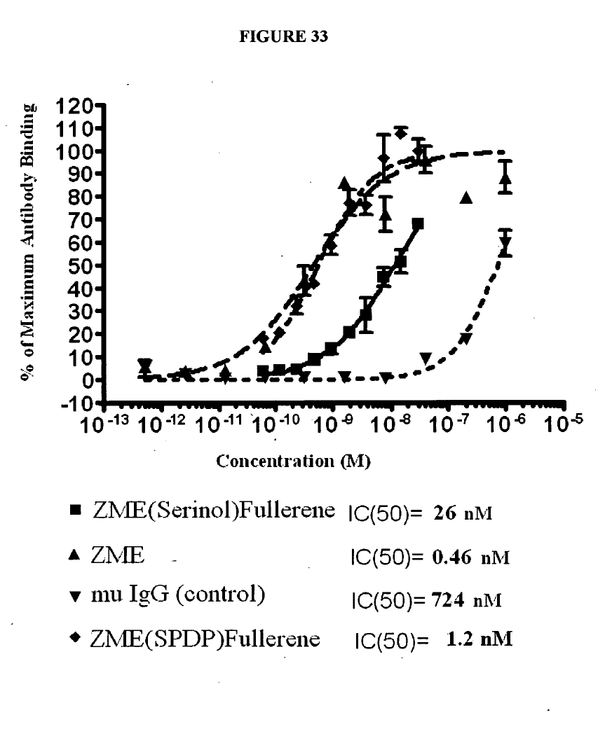

ELISA binding curves using antigen-positive cells as targets gave mid-points of 1.2 nM for the C₆₀-SPDP-(ZME-018) immunoconjugate, 26 nM for the C₆₀-Ser-(ZME-018) immunoconjugate (these values were adjusted by a factor of 2 after determining a more accurate ZME-018 concentration using a standard curve, and 724 nM for a non-specific, isotype-matched murine IgG antibody used as a control (FIG. 33). Amazingly, the C₆₀-SPDP-(ZME-018) conjugate demonstrated binding midpoints nearly identical to the non-conjugated ZME-018 antibody (mid-point of 0.46 nm), even though 15% (by weight) of the immunoconjugate is fullerene. However, the non-covalently bound C₆₀-Ser-(ZME-018) conjugate, consisting of 26% (by weight) fullerene, exhibited a much lower affinity than C₆₀-SPDP-(ZME-018). Regardless, the C₆₀-Ser-(ZME-018) conjugate was still a factor of 30 more effective in binding the target than was the control.

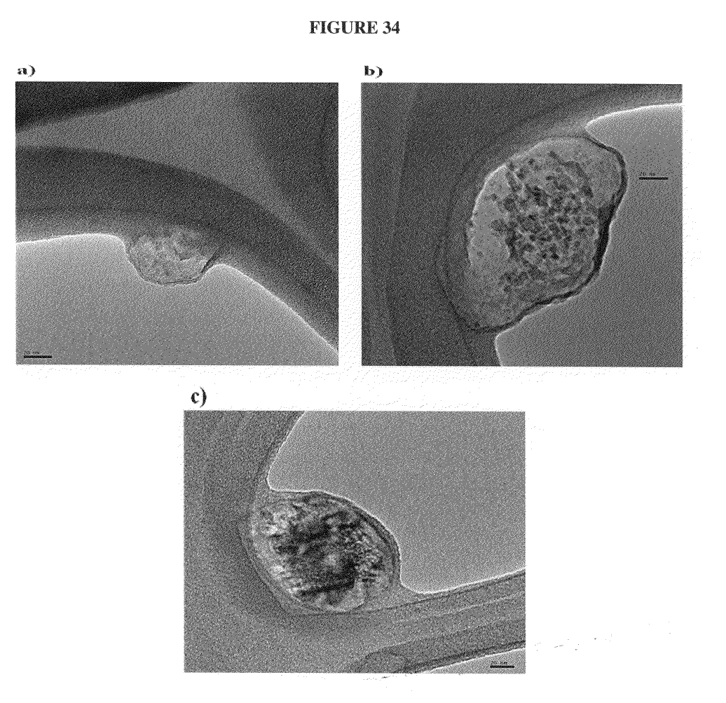

To visualize the two C₆₀-immunoconjugates, TEM images of both were obtained on a lacy carbon grid. Comparative images of the ZME-018 antibody and the immunoconjugates are shown in FIG. 34. The figure shows that the free antibody appears to have a clear globular structure ˜60 nm in diameter, whereas the image of the C₆₀-Ser and C₆₀-SPDP immunoconjugates are also globular, but 4-5 times larger in diameter. In addition, these immunoconjugate images reveal numerous dark spots scattered throughout the structure that can be attributed to small aggregates of C₆₀-Ser, ˜2-5 nm in diameter. The larger immunoconjugate sizes may reflect disruption of hydrogen bonding networks inside the antibody or some aggregation effect.

The above example confirms that covalent bond formation was not necessary to form immunoconjugates of water-soluble C₆₀ derivatives with an antibody, and that antibody to antigen binding was not significantly reduced for high C₆₀:antibody molar ratios (15:1). Further studies explored the cancer cell biology of these new C₆₀-immunoconjugates, as well as immunoconjugates derived from other fullerene-based nanostructures that have the potential for targeted imaging and therapy in medicine.

### Example 5

### Cell Internalization of Gd@C60-Immunoconjugates

Cell internalization studies were performed to determine the efficiency with which the cell-specific C₆₀-immunoconjugates internalize into melanoma cells. Antigen positive (A375m) cells were prepared in a 96-well plate (5000 cells/well) using Dulbecco's modified eagle medium. The cells were incubated overnight at 37° C., followed by addition of 100 μL/well of the C₆₀-immunoconjugates over various time frames. Incubation for 1, 4, 8, and 48 hr at 37° C. allowed for cell internalization to occur. At the zero point, the media was removed and each cell sample washed with DPBS to strip off any non-internalized C₆₀ immunoconjugate. Cells were then detached from the bottom of the plate and lysed in order to determine if the C₆₀-immunoconjugates internalized into the cells. Triplet-triplet absorption was once again implemented as a qualitative measure of C₆₀ cell internalization. Unfortunately, attempts at observing C₆₀ in the lysed cell solution by triplet-triplet absorption proved unproductive, showing no characteristic C₆₀ triplet-triplet bands. It was concluded that the sensitivity of triplet-triplet absorption spectrum was insufficient to detect the C₆₀-materials at concentrations <20 nM, which was the approximate amount of C₆₀ expected to internalize into the melanoma cells.

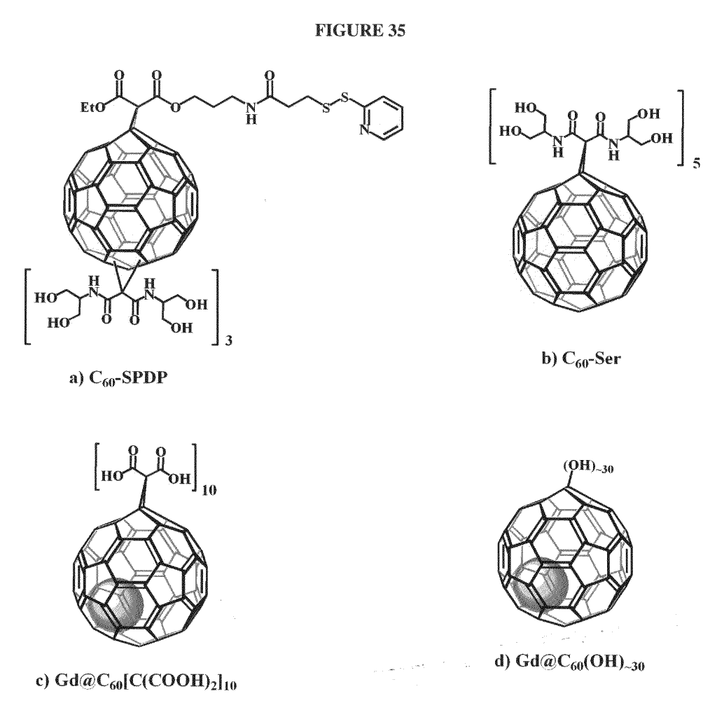

An alternative method that has shown sensitivity in the nM range is inductively-coupled plasma mass spectrometry (ICP-MS), with previous concentration determinations of several elements in water or waste extracts of digests <20 nM range, which was within the concentration range of C₆₀ expected to internalize into the cells. However, this method required an element other than carbon to detect. Fortunately, great strides have been made in the preparation and purification of water-soluble gadofullerenes, Gd@C₆₀[C(COOH)₂]₁₀ (Gd—COOH, FIG. 35c) and Gd@C₆₀(OH)₃₀ (Gd—OH, FIG. 35d), which were implemented to monitor the amount of C₆₀ internalized into the A375m melanoma cells.

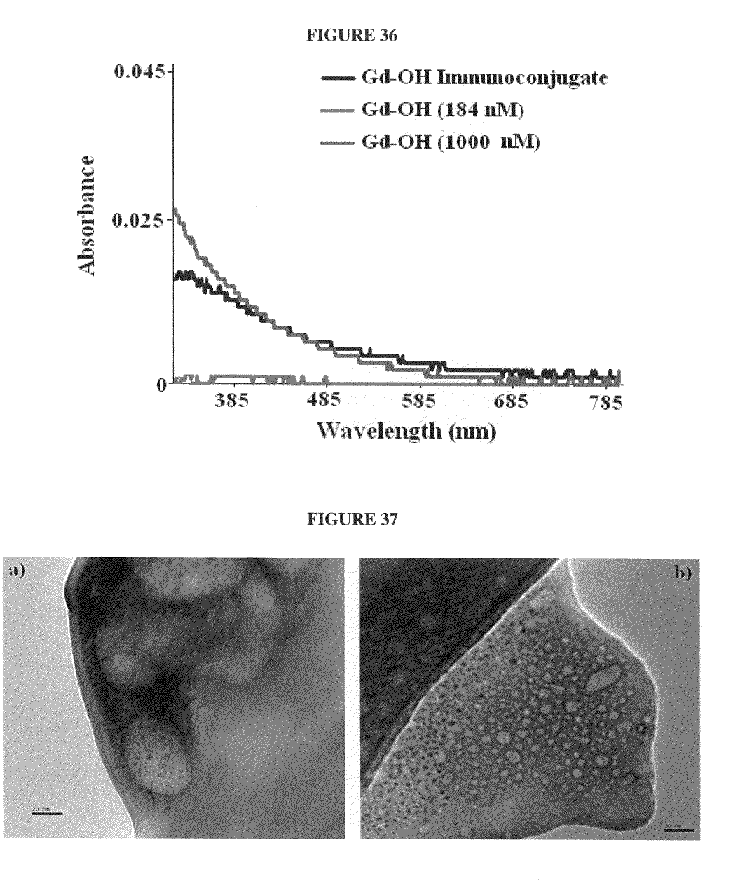

Immunoconjugates of Gd@C₆₀(OH)₃₀ and Gd@C₆₀[C(COOH)₂]₁₀ were prepared in similar fashion as the C₆₀-based immunoconjugates. Using ICP-atomic emission spectroscopy (ICP-AE), the Gd—OH and Gd—COOH concentrations in the immunoconjugates were determined to be 180 nM and 47 nM. Bio-Rad protein assays then determined the antibody concentration in the Gd—OH-(ZME-018) to be 875 nM (for a 1:5 molar ratio of Gd—OH:antibody) and 624 nM for the Gd—COOH-(ZME-018) (for a 1:13 molar ratio of Gd—COOH:antibody). The amount of Gd@C₆₀ was, therefore, significantly less than for empty C₆₀ in the C₆₀-immunoconjugates prepared above. This may be attributed to greater aggregation of the Gd@C₆₀-derivatives, when compared to the empty C₆₀ derivatives. This greater degree of aggregation stems from the inability of Gd@C₆₀ aggregates to thoroughly separate at the salt concentration and low temperature utilized in the immunoconjugation.⁸⁹ It appears that the C₆₀-Ser and C₆₀-SPDP are able to disaggregate to greater extents under the conditions used for immunoconjugation. This implies that the Gd@C₆₀ immunoconjugates reported here actually contain mostly empty C₆₀ derivatives, C₆₀(OH), and C₆₀(COOH)ₓ, rather than Gd@C₆₀ materials. In fact, the initial Gd@C₆₀(OH)₃₀ and Gd@C₆₀(COOH)₁₀ samples used to prepare the immunoconjugates only contain 70% and 50% gadofullerene, respectively, with the remainder of the sample being empty C₆₀ derivatives. In order to test this hypothesis, UV-vis spectra (FIG. 36) of the Gd—OH-(ZME-018) immunoconjugate was obtained and compared with the Gd@C₆₀(OH)₃₀ spectra at 180 nM (value of Gd³⁺ in immunoconjugate as determined by ICP-AE). It was observed that the Gd—OH immunoconjugate spectrum exhibited an absorbance from 280-600 nm. In contrast, Gd@C₆₀(OH)₃₀ diluted to 184 nM (concentration of Gd³⁺ in the immunoconjugate) displayed no absorbance over the same range. The most reasonable explanation for this observance was that the absorbance from the Gd—OH immunoconjugate is due to empty C₆₀ derivatives within the ZME-018 mAb. For comparison, a 1 μM Gd@C₆₀(OH)₃₀ solution was prepared, which shows an absorbance spectra similar to the immunoconjugate. This suggests that the Gd—OH immunoconjugate consisted of an abundance of empty C₆₀ material compared to Gd@C₆₀(OH)₃₀. However, attempts to quantify the amount of empty C₆₀ using UV-vis is not possible using a standard curve from Gd@C₆₀(OH)₃₀ samples.

TEM images of the Gd@C₆₀ immunoconjugates were acquired in order to visualize the Gd—OH and Gd—COOH interaction with the ZME-018 mAb (FIG. 37). Similar to the C₆₀-based immunoconjugates, the ZME-018 mAb has increased in size and contains aggregates of the C₆₀-based nanomaterials as seen by the uniform black spots. These results show that the Gd@C₆₀ materials display similar interactions (to a smaller extent) as the C₆₀-SPDP and C₆₀-Ser derivatives with ZME-018, but that conjugation conditions must be optimized in order to increase the amount of Gd@C₆₀-derivatives in any of the ZME-018 mAb conjugates.

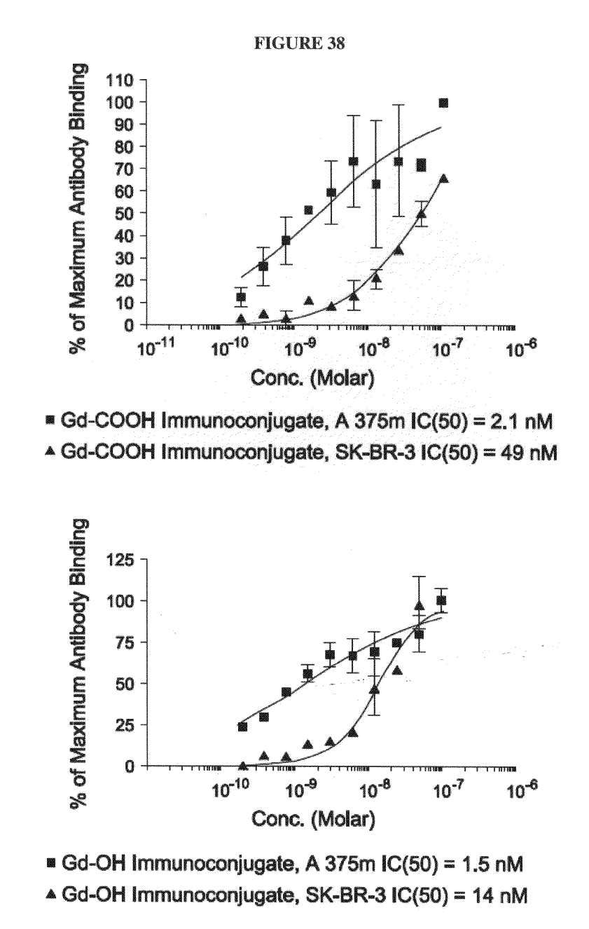

Cell binding affinity was once again evaluated by calculating IC(50) values from ELISA plots. Similarly to the C₆₀-immunoconjugates, dry cell A375m (antigen+) cells were utilized. However, to better understand binding efficiencies, SK-BR-3 (antigen−) cells were also used for comparison with the antigen positive cells. The Gd—OH-(ZME-018) and Gd—COOH-(ZME-018) immunoconjugates ELISA biding curves and IC(50) were each analyzed with both cell lines (FIG. 38). The IC(50) values and hence binding efficiencies to the A375m cells for the Gd—COOH immunoconjugate was 2.1 nM and Gd—OH immunoconjugate was 1.5 nM. This is practically identical to non-conjugated ZME-018, which demonstrated a IC(50) value of 3.6 nM (plot not shown). When juxtaposed with the SK-BR-3 antigen negative cell line, which showed the IC(50) values as 14 nM for the Gd—OH immunoconjugate (nine times less efficient) and 49 nM for the Gd—COOH-immunoconjugate (23 times less efficient), it is evident that the retained cell specificity of the Gd@C₆₀-based immunoconjugates is a major step forward for the future development of FIT.

Cell internalization studies for the Gd@C₆₀-immunoconjugates were performed in a manner similar to that for the C₆₀-immunoconjugates. Deviations from the previous method occurred when lysing the cell. Instead of using a lysis buffer, cells were removed from the plate and placed in a scintillation vial. Approximately 1.5 mL of 25% chloric acid was added to the vial and heated to 90° C. for 30 min in order to consume the cells and destroy the C₆₀ cage around gadolinium. After cooling, 10 mL of 2% nitric acid was then added as the matrix utilized for ICP-MS.

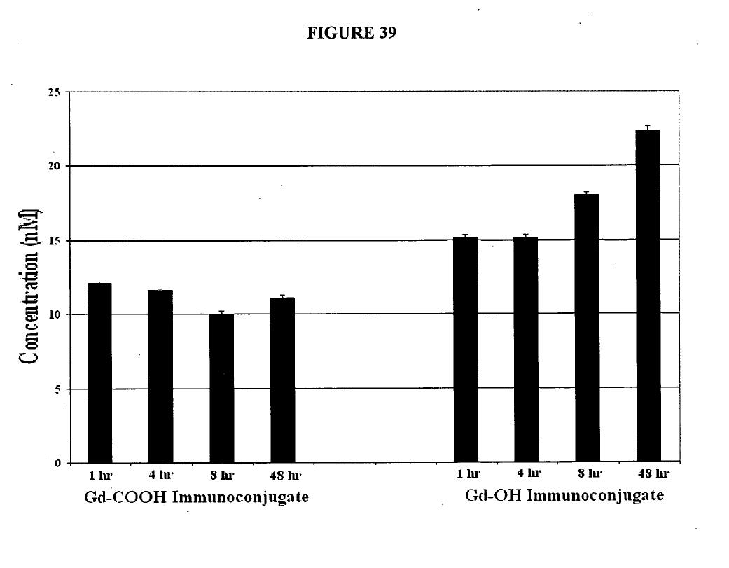

The Gd³⁺ concentration was determined in triplicate using ICP-MS for cell internalization studies using both Gd—OH-(ZME-018) and Gd—COOH-(ZME-018) (FIG. 39). Standard deviations were determined for the three aliquots of one cell internalization sample at each time point. This deviation only shows the accuracy of the ICP-MS. In order to obtain more accurate Gd³⁺ internalization data, a greater number of separate cell internalizations must be performed and analyzed. Regardless, for the Gd—COOH-(ZME-018) conjugate, it is clear that the amount of Gd—COOH immunoconjugate that internalizes remains relatively constant over time, between 10-13 nM. However, the Gd—OH immunoconjugate appears to exhibit a slight increase in delivery of Gd—OH, with the concentration increasing from 15 to 23 nM over time from 1 to 48 hr. This contrast could be attributed to much greater Gd—OH concentration found in the Gd—OH-(ZME-018) immunoconjugate (180 nM vs. 47 nM). It is reasonable for the Gd—OH-(ZME-018) to internalize Gd³⁺ ion to a greater extent due to its higher Gd³⁺ concentration in the immunoconjugate.

These initial internalization experiments demonstrate the feasibility of utilizing ICP-MS for determining [Gd³⁺] at very low concentrations after cell internalization of Gd—OH and Gd—COOH immunoconjugates into A375m cells. For comparison, attempts to internalize the Gd@C₆₀-immunoconjugates into TXM-1 antigen negative cells were performed. These internalizations showed no internalization of the Gd³⁺ into the TXM-1 cells, demonstrating that the Gd@C₆₀-immunoconjugates retained their cell specific properties, as well as verifying that cell internalization into the A375m cells was successful. A second study, which analyzed both the cells and the exo-cellular wash solution, revealed that approximately 20% of Gd—OH immunoconjugate are internalized into cells, while the other 80% eluted with the wash solution. These results suggest that Gd@C₆₀-based immunoconjugates do internalize into cells and that optimization of this internalization will be needed for the future development of FIT.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A targeted carbon nanostructure comprising: a Cₙ, a cross-linker, and a targeting agent, wherein Cₙ refers to a fullerene moiety or nanotube comprising n carbon atoms.

2. The targeted carbon nanostructure of claim 1 wherein the Cₙ is substituted with malonate groups, serinol malonates, groups derived from malonates, serinol groups, carboxylic acid, polyethyleneglycol (PEG), or combinations thereof.

3. The targeted carbon nanostructure of claim 1 wherein the cross linker is N-succinimidyl-3-(2-pyridyldithio)-propionate (SPDP), serinol, or combinations thereof.

4. The targeted carbon nanostructure of claim 1 wherein the targeting agent comprises an antibody.

5. The targeted carbon nanostructure of claim 1 wherein the Cₙ is a buckminsterfullerene, gadofullerene, single walled carbon nanotube (SWNT), or an ultra-short carbon nanotube.

6. The targeted carbon nanostructure of claim 1 further comprising a contrast agent.

7. A method for imaging comprising: contacting a targeted carbon nanostructure and a cell; allowing the cell to internalize the carbon nanostructure; and detecting the presence of internalized carbon nanostructures.

8. The method of claim 7 wherein the cell is a melanoma cell.

9. The method of claim 7 wherein the targeted carbon nanostructure comprises a Cₙ, a cross linker, a contrasting agent, and a targeting agent.
